(19) United States
(12) Patent Application Publication
(10) Pub. No.: US 2024/0074980 A1
(43) Pub. Date: Mar. 7, 2024

(54) FORMULATIONS FOR NON-PARENTERAL ADMINISTRATION OF EXOSOMES

(71) Applicant: Vitabolus Inc., La Jolla, CA (US)

(72) Inventor: Aline BETANCOURT, La Jolla, CA (US)

(21) Appl. No.: 18/256,021

(22) PCT Filed: Dec. 10, 2021
(86) PCT No.: PCT/US2021/062827

§ 371 Date: Jun. 5, 2023

Related U.S. Application Data
(60) Provisional application No. 62/124,312, filed on Dec. 15, 2014.

(51) Int. Cl. A61K 9/48; A61K 9/00; A61K 35/28; A61K 47/32; A61K 47/38; A61P 1/00; A61P 29/00

(52) U.S. Cl. CPC A61K 9/4816; A61K 9/0053; A61K 35/28; A61K 47/32; A61K 47/38; A61P 1/00; A61P 29/00

(57) ABSTRACT

Described herein is a composition for oral delivery of mammalian exosomes, the composition comprising a plurality of mammalian exosomes admixed with a matrix and surrounded by an enteric coating, the matrix comprising a matrix comprising about 0.5% to about 3.0% methyl cellulose and about 0.5% to about 3.0% alginate gel. Also described herein is a composition for rectal or vaginal delivery of mammalian exosomes, the composition comprising a lipophilic or hydrophilic coating surrounding a plurality of mammalian exosomes admixed with a matrix in solid form, the matrix comprising a matrix comprising about 0.5% to about 3.0% methyl cellulose and about 0.5% to about 3.0% alginate gel.

### CROSS REFERENCE

This application claims the benefit of previously filed U.S. Provisional Application Ser. No. 63/124,312 filed on Dec. 11, 2020, which is incorporated by reference herein in its entirety.

### SUMMARY

Described herein are formulations and compositions useful for the non-parenteral administration of exosomes.

In one aspect described herein is a composition for oral delivery of mammalian exosomes, the composition comprising a plurality of mammalian exosomes admixed with a matrix and surrounded by an enteric coating, the matrix comprising a matrix comprising about 0.1% to about 5.0% methyl cellulose and about 0.1% to about 5.0% alginate gel. In certain embodiments, the matrix comprises about 0.5% to about 1.0% methyl cellulose and about 0.5% to about 1.0% alginate gel. In certain embodiments, the matrix comprises about 0.25% to about 0.75% methyl cellulose and about 0.25% to about 0.75% alginate gel. In certain embodiments, the matrix comprises about 0.5% to about 3.0% methyl cellulose and about 0.5% to about 3.0% alginate gel. In certain embodiments, the matrix comprises about 1.0% to about 2.0% methyl cellulose and about 1.0% to about 2.0% alginate gel. In certain embodiments, the matrix comprises about 1.3% to about 1.8% methylcellulose and about 1.3% to about 1.8% alginate gel. In certain embodiments, the matrix comprises about 1.5% methylcellulose and about 1.5% alginate gel. In certain embodiments, the matrix comprises a ratio of methyl cellulose to alginate gel of about 1:1. In certain embodiments, the exosomes are mesenchymal stem cell exosomes or hematopoietic stem cell exosomes. In certain embodiments, the exosomes are hematopoietic stem cell exosomes. In certain embodiments, the exosomes are mesenchymal stem cell exosomes. In certain embodiments, the mesenchymal stem cells are type I mesenchymal stem cells. In certain embodiments, the mesenchymal stem cells are type II mesenchymal stem cells. In certain embodiments, the exosomes comprise a protein or nucleic acid selected from the list consisting of CD81, CD63, CD9, HSP60, HSP70, HSP90, VEGF, PGE2, and combinations thereof. In certain embodiments, the exosomes comprise a nucleic acid selected from the list consisting of miRNA Let7, 155,146, 122, and combinations thereof. In certain embodiments, the mammalian exosomes possess a diameter from about 30 nanometers to about 150 nanometers. In certain embodiments, the mammalian exosomes are human exosomes. In certain embodiments, the mammalian exosomes are canine, feline, bovine, equine, ovine, or porcine exosomes. In certain embodiments, the enteric coating comprises methyl methacrylate-methacrylic acid copolymer (1:1). In certain embodiments, the enteric coating consists essentially of methyl methacrylate-methacrylic acid copolymer (1:1). In certain embodiments, the composition comprises at least 100 billion exosomes. In certain embodiments, the composition comprises from at least 100 billion exosomes to no more than 2000 billion exosomes.

Also described herein in another aspect is a composition for non-parenteral and/or rectal or vaginal delivery of mammalian exosomes, the composition comprising a lipophilic or hydrophilic coating surrounding a plurality of mammalian exosomes admixed with a matrix in solid form, the matrix comprising a matrix comprising about 0.5% to about 3.0% methyl cellulose and about 0.5% to about 3.0% alginate gel. In certain embodiments, the matrix comprises about a matrix comprises about 1.0% to about 2.0% methyl cellulose and about 1.0% to about 2.0% alginate gel. In certain embodiments, the matrix comprises about 1.3% to about 1.8% methylcellulose and about 1.3% to about 1.8% alginate gel. In certain embodiments, the matrix comprises about 1.5% methylcellulose and about 1.5% alginate gel. In certain embodiments, the matrix comprises a ratio of methyl cellulose to alginate gel of about 1:1. In certain embodiments, the exosomes are mesenchymal stem cell exosomes or hematopoietic stem cell exosomes. In certain embodiments, the exosomes are hematopoietic stem cell exosomes. In certain embodiments, the exosomes are mesenchymal stem cell exosomes. In certain embodiments, the mesenchymal stem cells are type I mesenchymal stem cells. In certain embodiments, the mesenchymal stem cells are type II mesenchymal stem cells. In certain embodiments, the exosomes comprise a protein or nucleic acid selected from the list consisting of CD81, CD63, CD9, HSP60, HSP70, HSP90, VEGF, PGE2, and combinations thereof. In certain embodiments, the exosomes comprise a nucleic acid selected from the list consisting of miRNA Let7, 155,146, 122, and combinations thereof. In certain embodiments, the mammalian exosomes possess a diameter from about 30 nanometers to about 150 nanometers. In certain embodiments, the mammalian exosomes are human exosomes. In certain embodiments, the mammalian exosomes are canine, feline, bovine, equine, ovine, or porcine exosomes. In certain embodiments, the composition is formulated for rectal delivery. In certain embodiments, the composition is formulated for vaginal delivery. In certain embodiments, the formulation does not swell upon contact with an aqueous medium. In certain embodiments, the lipophilic or hydrophilic coating dissolves at a normal body temperature, or at 36° C. and above. In certain embodiments, the lipophilic or hydrophilic coating consists essentially of cocoa butter, coconut oil, glycerated gelatin, hydrogenated oil, polyethylene glycol (PEG), a fatty acid ester of PEG, or Gattefosse lipophilic bases. In certain embodiments, the lipophilic or hydrophilic coating comprises cocoa butter, coconut oil, glycerated gelatin, hydrogenated oil, polyethylene glycol (PEG), a fatty acid ester of PEG, or Gattefosse lipophilic bases. In certain embodiments, the enteric coating consists essentially of methyl methacrylate-methacrylic acid copolymer (1:1). In certain embodiments, the composition comprises at least 100 billion exosomes. In certain embodiments, the composition comprises from at least 100 billion exosomes to no more than 2000 billion exosomes. In certain embodiments, greater than 40% or more intact exosomes compared to the starting amount are recovered from the composition after a 2 hour incubation at pH 1.2. In certain embodiments, greater than 50% or more intact exosomes compared to the starting amount are recovered from the composition after a 2 hour incubation at pH 1.2. In certain embodiments, greater than 60% or more intact exosomes compared to the starting amount are recovered from the composition after a 2 hour incubation at pH 1.2.

The compositions described herein in one aspect may be used in a method of treating an inflammatory or autoimmune disorder in an individual comprising administering the composition, non-parenterally, orally, vaginally, or rectally to an individual. In certain embodiments, the individual is a human individual. In certain embodiments, the individual is a dog, a cow, a cat, a pig, a horse, or a sheep. In certain embodiments, the inflammatory or autoimmune disorder is a gastrointestinal inflammatory or autoimmune disorder. In certain embodiments, the gastrointestinal inflammatory or autoimmune disorder comprises inflammatory bowel disease or Crohn's disease. In certain embodiments, the gastrointestinal inflammatory or autoimmune disorder comprises Crohn's disease. Also described herein is a method of reducing neutrophil accumulation or activation of neutrophils in the gastrointestinal tract of an individual comprising administering the composition described herein to an individual.

### BRIEF DESCRIPTION OF THE DRAWINGS

The novel features described herein are set forth with particularity in the appended claims. A better understanding of the features and advantages of the features described herein will be obtained by reference to the following detailed description that sets forth illustrative examples, in which the principles of the features described herein are utilized, and the accompanying drawings of which:

FIG. 1 illustrates a representative scheme of oral exosome delivery capsules. The therapeutic mammalian exosomes would be properly sourced and manufactured, loaded or admixed in a matrix, packaged into a capsule and finalized with enteric coating(s) in the single capsule monophasic delivery scheme on the top. In another embodiment, the therapeutic mammalian exosomes would be manufactured, loaded or admixed in a matrix as before being packaged into one capsule. Subsequently, a second dose of exosomes would be admixed in a matrix and packaged into a capsule that would next be loaded within the first capsule as shown in the capsule in capsule biphasic delivery scheme on the bottom.

FIG. 2 illustrates a representative, non-limiting, scheme of oral delivery of the exosome loaded capsules and their proposed deployment to the ileum and colon. The therapeutic mammalian exosomes in the single capsule monophasic delivery (left) or the capsule in capsule biphasic delivery (right) would be taken orally and travel past the mouth, esophagus, and duodenum with the uncoating of the protective enteric coating and exosome deployment occurring at the targeted ileum pH 7.3-8 and subsequent colon pH 5.5-6.5 as shown.

FIG. 3 illustrates a representative scheme of rectal or vaginal exosome delivery capsules. The therapeutic mammalian exosomes would be manufactured, loaded or admixed in a matrix, packaged into a capsule and finalized with enteric coating(s) in the single capsule monophasic delivery scheme on the top. In another embodiment, the therapeutic mammalian exosomes would be properly sourced and manufactured, loaded or admixed in a matrix as before being packaged into one capsule. Subsequently, a second dose of exosomes would be admixed in a matrix and packaged into a capsule that would next be loaded within the first capsule as shown in the capsule in capsule biphasic delivery scheme on the bottom.

FIG. 4 illustrates a representative, non-limiting, scheme of rectal and or vaginal delivery of the exosome loaded capsules and their proposed deployment to the ileum and colon. The therapeutic mammalian exosomes in the single capsule monophasic delivery (left) or the capsule in capsule biphasic delivery (right) would be taken orally and travel past the mouth, esophagus, and duodenum with the uncoating of the protective hydrophilic or lipophilic coating and exosomes deployment occurring at the targeted vaginal pH 3.8-4.7 or rectal pH 5.5-8.0 and subsequent colon pH 5.5-6.5 as shown.

FIG. 5 shows a production scheme for the delivery systems described herein.

FIG. 6 shows that exosome particles are released from oral formulated capsules after reaching neutral pH. Exosomes were derived from adult human mesenchymal stem cells, loaded into an oral formulation with enteric coating and incubated at the indicated pH. FIG. 6A shows a heat map of data. FIG. 6B shows the counts of exosomes expressing CD63, CD9, or CD81 recovered from each pH incubation. FIG. 6C shows immunofluorescence microscopy showing intact exosomes are recovered.

FIG. 7 shows that exosome particles are only released from oral formulated capsules after incubation in simulated gastric or intestinal fluid. FIG. 7A shows a heat map of data. FIG. 7B shows the counts of exosomes expressing CD63, CD9, or CD81 recovered from simulated gastric fluid (SIG) or simulated intestinal fluid (SIF). FIG. 7C shows immunofluorescence microscopy showing intact exosomes are recovered.

FIG. 8 shows that oral exosome therapy attenuates IBD colitis in a murine model. FIG. 8A shows the experimental set up. FIG. 8B shows scoring from the in vivo model.

FIG. 9 shows that recovered exosomes retain anti-inflammatory potency.

### DETAILED DESCRIPTION

Described herein is a composition for non-parenteral delivery of mammalian exosomes, the composition comprising a plurality of mammalian exosomes admixed with a matrix, the matrix comprising alginate gel. In certain embodiments, the composition is for oral delivery. In certain embodiments, the composition releases the mammalian exosome at a pH of about 6.4, 6.6, 6.8, or 7.0 or greater.

Described herein is a composition for non-parenteral delivery of mammalian exosomes, the composition comprising a plurality of mammalian exosomes admixed with a matrix, the matrix comprising methyl cellulose. In certain embodiments, the composition is for oral delivery. In certain embodiments, the composition releases the mammalian exosome at a pH of about 6.4, 6.6, 6.8, or 7.0 or greater.

Described herein is a composition for non-parenteral delivery of mammalian exosomes, the composition comprising a plurality of mammalian exosomes admixed with a matrix and surrounded by an enteric coating, the matrix comprising alginate gel. In certain embodiments, the composition is for oral delivery. In certain embodiments, the composition releases the mammalian exosome at a pH of about 6.4, 6.6, 6.8, or 7.0 or greater.

Described herein is a composition for non-parenteral delivery of mammalian exosomes, the composition comprising a plurality of mammalian exosomes admixed with a matrix and surrounded by an enteric coating, the matrix comprising methyl cellulose. In certain embodiments, the composition is for oral delivery. In certain embodiments, the composition releases the mammalian exosome at a pH of about 6.4, 6.6, 6.8, or 7.0 or greater.

Described herein is a composition for non-parenteral delivery of mammalian exosomes, the composition comprising a plurality of mammalian exosomes admixed with a matrix and surrounded by an enteric coating, the matrix comprising a matrix comprising methyl cellulose and alginate gel. In certain embodiments, the composition is for oral delivery. In certain embodiments, the composition releases the mammalian exosome at a pH of about 6.4, 6.6, 6.8, or 7.0 or greater.

Described herein is a composition for oral delivery of mammalian exosomes, the composition comprising a plurality of mammalian exosomes admixed with a matrix and surrounded by an enteric coating, the matrix comprising a matrix comprising about 0.1% to about 5.0% methyl cellulose and about 0.1% to about 5.0% alginate gel.

In the following description, certain specific details are set forth in order to provide a thorough understanding of various embodiments. However, one skilled in the art will understand that the embodiments provided may be practiced without these details. Unless the context requires otherwise, throughout the specification and claims which follow, the word “comprise” and variations thereof, such as, “comprises” and “comprising” are to be construed in an open, inclusive sense, that is, as “including, but not limited to.” As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. Further, headings provided herein are for convenience only and do not interpret the scope or meaning of the claimed embodiments.

Compositions recited herein can “consist essentially” of the named components, which excludes other active ingredients then the named components, but may include other diluents, carriers, and excipients which do not alter the fundamental properties of the composition (often included to contribute to administrability or stability solution).

As described herein “non-parenteral” refers to any route of administration that is enteral including through the oral, rectal, or nasal pathways. Non-parenteral as described herein also refers to vaginal administration.

As used herein the term “about” refers to an amount that is near the stated amount by 10% or less.

As used herein the term “individual,” “patient,” or “subject” refers to individuals diagnosed with, suspected of being afflicted with, or at-risk of developing at least one disease for which the described compositions and method are useful for treating. In certain embodiments the individual is a mammal. In certain embodiments, the mammal is a mouse, rat, rabbit, dog, cat, horse, cow, sheep, pig, goat, llama, alpaca, or yak. In certain embodiments, the individual is a human.

The terms “polypeptide” and “protein” are used interchangeably to refer to a polymer of amino acid residues, and are not limited to a minimum length. Polypeptides, including the provided antibodies and antibody chains and other peptides, e.g., linkers and binding peptides, may include amino acid residues including natural and/or non-natural amino acid residues. The terms also include post-expression modifications of the polypeptide, for example, glycosylation, sialylation, acetylation, phosphorylation, and the like. In some aspects, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification.

As described herein “hematopoietic stem cells” and grammatical equivalents refer to the stem cells that give rise to other blood cells including red blood cells; platelets; lymphoid cells, such as, T cells, B cells; and myeloid cell, such as monocytes, macrophages, eosinophils, basophils, mast cells, and neutrophils. Hematopoietic stem cells per this disclosure are cells that can give rise to at least lymphoid and myeloid cells. Common surface markers for hematopoietic stem cells comprise CD34. Hematopoietic stem cells can also comprise surface markers such as EPCR and CD90. Hematopoietic stem cells can be identified by any one or more of CD34, EPCR, and CD90. Such surface positivity can be identified for by example immunohistochemistry or flow cytometry methods. Hematopoietic stem cells can suitably be isolated from bone-marrow, umbilical cord blood, and the peripheral blood. Hematopoietic stem cells can be cultured using methods known in the art. See e.g., Frisch et al. “Hematopoietic Stem Cell Cultures and Assays.” Methods Mol Biol. 2014; 1130: 315-324.

As described herein “mesenchymal stem cells” and grammatical equivalents refer to a stem cell originally derived from the mesenchyme. The term refers to a cell which is capable of differentiating into at least two or more of an osteoblast, a chondrocyte, an adipocyte, or a myocyte. MSCs may be isolated from any type of adult tissue. Typically, MSCs are isolated from bone marrow, adipose tissue, umbilical cord, or peripheral blood. MSCs can be divided into proinflammatory MSC type I and anti-inflammatory MSC type II as described in Waterman et al. “A New Mesenchymal Stem Cell (MSC) Paradigm: Polarization into a Pro-Inflammatory MSC1 or an Immunosuppressive MSC2 Phenotype.” PLoS One. 2010; 5(4): e10088. Methods of induction of various MSC types are described in for example: US20140017787A1 and US20160097038A1.

As described herein “exosomes” abbreviated (EV) and grammatical equivalents refer to membrane-bound extracellular vesicles that are produced by many eukaryotic cells. These vesicles can contain proteins and nucleic acids such as DNAs, RNAs, mRNAs, or miRNAs. Exosomes can be produced by many different methods from cells in culture. Such methods comprise for example centrifugation techniques and kits commercially available. See e.g., Lane et al. “Analysis of exosome purification methods using a model liposome system and tunable-resistive pulse sensing.” Scientific Reports volume 5, Article number: 7639 (2015); Li et al. “Progress in exosome isolation techniques.” Theranostics. 2017; 7(3): 789-804; WO2019035880A1; and WO2019231562A1.

In certain embodiments, EVs according to this disclosure possess a diameter of about 25 nm to about 150 nm. In certain embodiments, EVs according to this disclosure possess a diameter of about 25 nm to about 50 nm, about 25 nm to about 75 nm, about 25 nm to about 100 nm, about 25 nm to about 125 nm, about 25 nm to about 150 nm, about 50 nm to about 75 nm, about 50 nm to about 100 nm, about 50 nm to about 125 nm, about 50 nm to about 150 nm, about 75 nm to about 100 nm, about 75 nm to about 125 nm, about 75 nm to about 150 nm, about 100 nm to about 125 nm, about 100 nm to about 150 nm, or about 125 nm to about 150 nm. In certain embodiments, EVs according to this disclosure possess a diameter of about 25 nm, about 50 nm, about 75 nm, about 100 nm, about 125 nm, or about 150 nm. In certain embodiments, EVs according to this disclosure possess a diameter of at least about 25 nm, about 50 nm, about 75 nm, about 100 nm, or about 125 nm. In certain embodiments, EVs according to this disclosure possess a diameter of at most about 50 nm, about 75 nm, about 100 nm, about 125 nm, or about 150 nm.

Recovery of exosomes form the compositions described herein refers to the recovery after a stated event, incubation etc., compared to a number of exosomes initially included in the composition. Established technologies to determine (average) EV sizes and concentrations are electron microscopy (EM), dynamic light scattering (DLS), nanoparticle tracking analysis (NTA), resistive pulse sensing, and fluorescence correlation spectroscopy. For the purposes of this disclosure particle number or size is measured either by standard flow cytometry or high resolution flow cytometry, depending on the size of the exosome. See e.g., van der Vlist E J, Nolte-'T Hoen E N, Stoorvogel W, et al. Fluorescent labeling of nano-sized vesicles released by cells and subsequent quantitative and qualitative analysis by high-resolution flow cytometry. Nat Protoc. 2012/06/23.2012; 7(7):1311-1326; van der Vlist E J, Nolte-'T Hoen E N, Stoorvogel W, et al. Fluorescent labeling of nano-sized vesicles released by cells and subsequent quantitative and qualitative analysis by high-resolution flow cytometry. Nat Protoc. 2012 Jun. 23. 2012; 7(7):1311-1326; respectively.

### Oral Delivery Systems

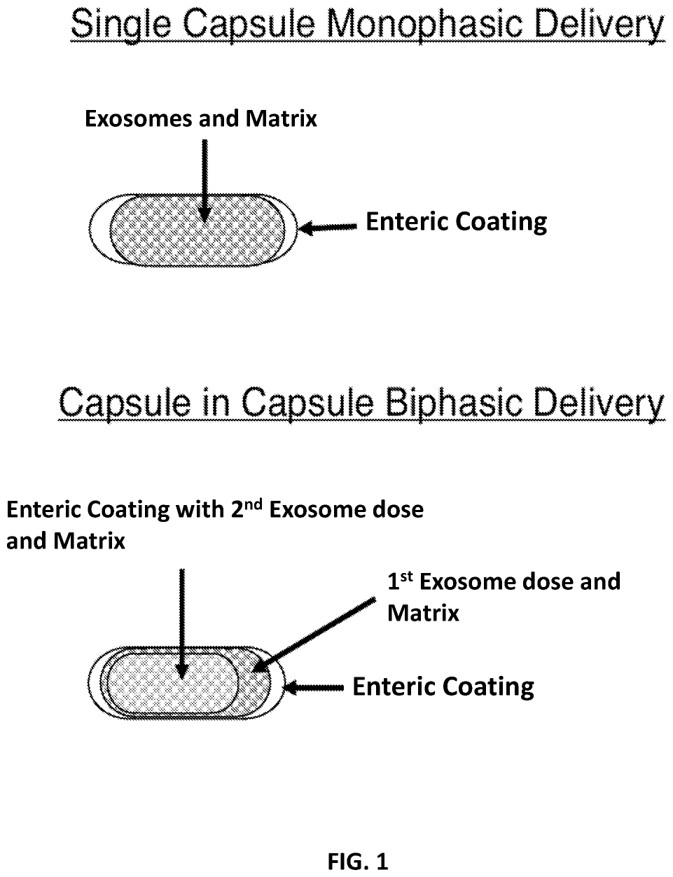

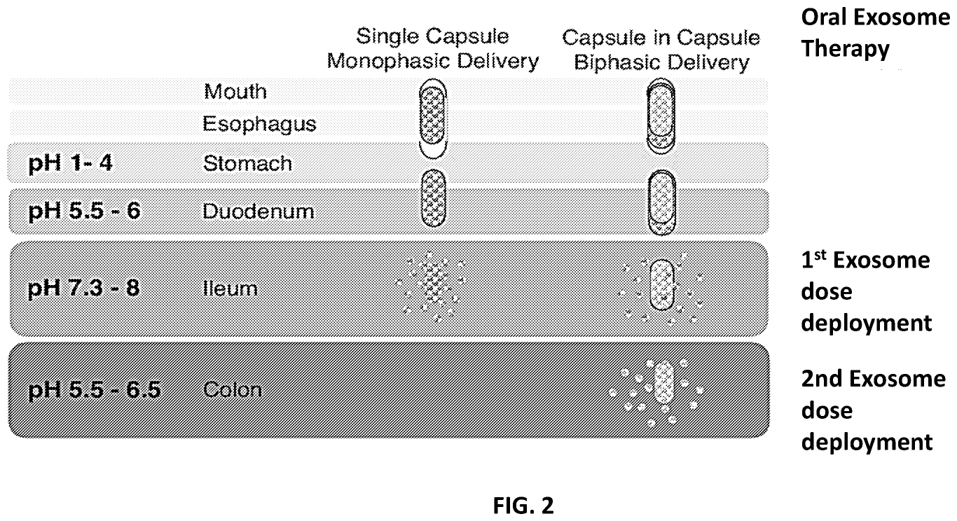

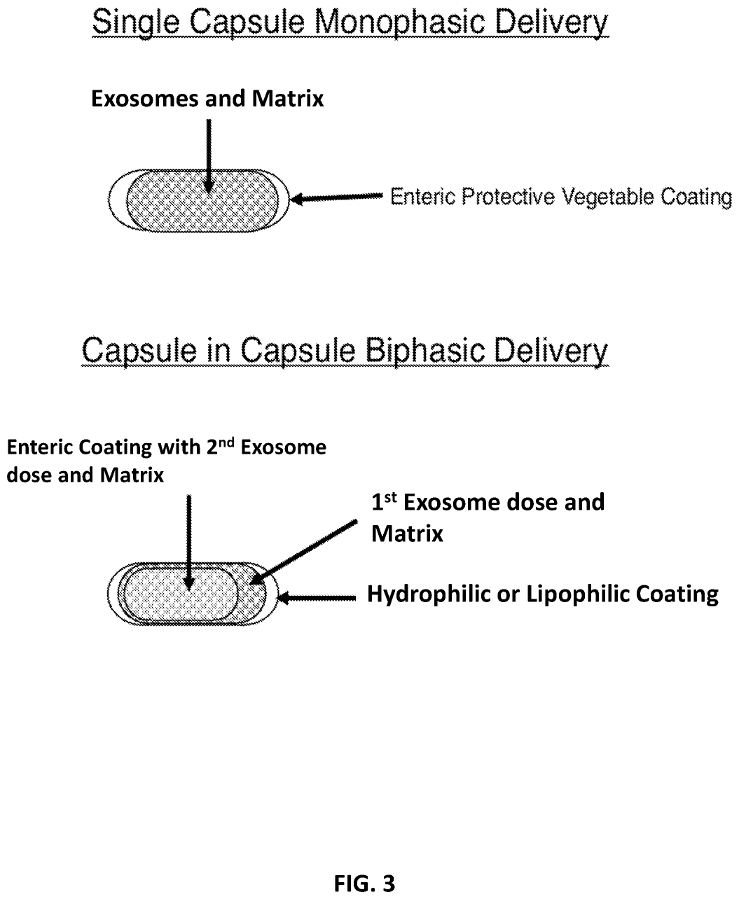

One aspect of the delivery systems described as shown in FIG. 1 is a monophasic delivery system. A monophasic delivery system comprises a plurality of exosomes and a pH sensitive enteric coating. Another aspect of the delivery systems described as shown in FIG. 1 is a biphasic delivery system. A biphasic delivery system comprises a first plurality of exosomes enclosed in an enteric coating, and a second plurality of exosomes in an enteric coating. The first plurality of exosomes surrounds the second plurality of exosomes, such that the first plurality of exosomes is released at a first pH, leaving the second plurality of exosomes to be released at a second pH. As a result, a biphasic delivery system can deliver two different pluralities of exosomes to two different locations in the alimentary canal of an individual as shown in FIG. 2. In certain embodiments, the first and the second plurality of exosomes comprise the same type of cell. In certain embodiments, the first and the second plurality of exosomes comprise different types of exosomes. In certain embodiments, the exosomes are embedded in a matrix and then surrounded by the enteric coating. The matrix serves to protect the exosomes from osmotic stress, pH stress, and mechanical disruption prior to release at the intended delivery site, which can comprise any one or more of the ileum, jejunum, duodenum, or colon. Systems such as these are useful in delivering therapeutic exosomes to an individual by an non-parenteral and oral route. FIG. 3 provides an overview of a process for making and distributing the oral exosome compositions described herein.

### Rectal or Vaginal Delivery Systems

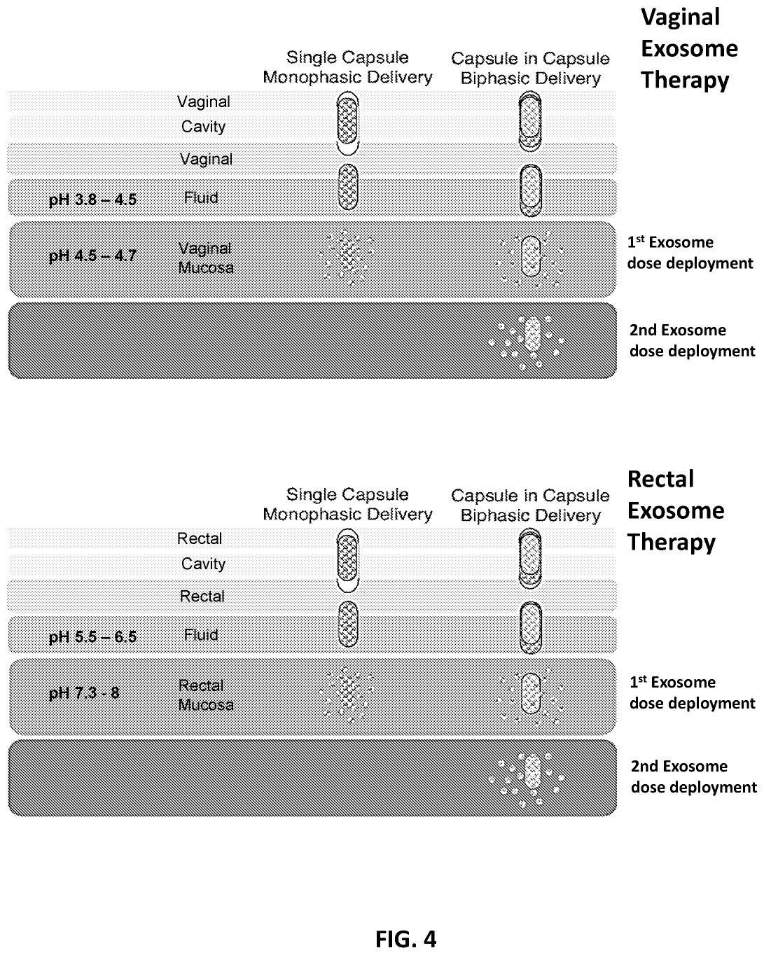

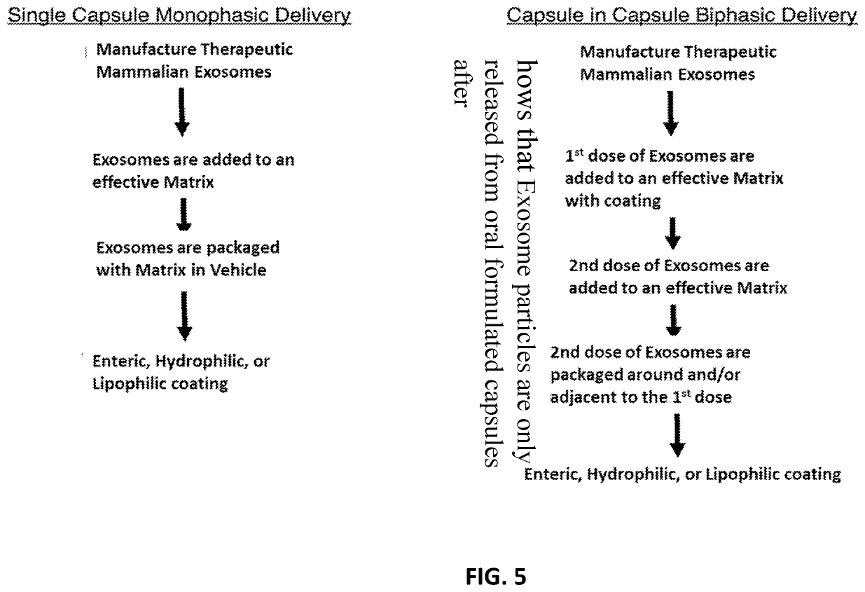

One aspect of the delivery systems described as shown in FIG. 3 is a monophasic delivery system. A monophasic delivery system comprises a preparation of exosomes and a pH sensitive lipophilic or hydrophilic coating. Another aspect of the delivery systems described as shown in FIG. 3 is a biphasic delivery system. A biphasic delivery system comprises a first preparation of exosomes enclosed in a lipophilic or hydrophilic coating, and a second preparation of exosomes in a lipophilic or hydrophilic coating. The first preparation of exosomes surrounds the second preparation of exosomes, such that the first preparation of exosomes is released at a first pH, leaving the second preparation of exosomes to be released at a second pH. As a result, a biphasic delivery system can deliver two different pluralities of exosome preparations to two different locations in the alimentary canal of an individual as shown in FIG. 4. In certain embodiments, the first and the second preparation of exosomes comprise the same type of exosome. In certain embodiments, the first and the second preparation of exosomes comprise different types of exosomes. In certain embodiments, the exosomes are embedded in a matrix and then surrounded by the lipophilic or hydrophilic coating. The matrix serves to protect the exosomes from osmotic stress, pH stress, and mechanical disruption prior to release at the intended delivery site, which can comprise any one or more of the colon, large intestine, small intestine, appendix, cecum, colon, rectum, duodenum, jejunum, ileum, or vagina. Systems such as these are useful in delivering therapeutic exosomes to an individual by a rectal or vaginal route. FIG. 3 provides an overview of a process for making and distributing the rectal or vaginal exosome preparation compositions described herein.

Pharmaceutically acceptable organic or inorganic carrier substances suitable for non-parenteral administration which do not deleteriously react with the components of the composition can also be included in the formulation. Suitable pharmaceutically acceptable carriers include, but are not limited to, water, salt solutions, alcohols, polyethylene glycols, gelatin, lactose, amylose, magnesium stearate, talc, silicic acid, viscous paraffin, hydroxymethylcellulose, polyvinylpyrrolidone, and the like, or any combination thereof. The formulations can be sterilized and, if desired, mixed with auxiliary agents, e.g., lubricants, preservatives, stabilizers, wetting agents, emulsifiers, salts for influencing osmotic pressure, buffers, colorings flavorings, aromatic substances, or any combination thereof.

The compositions of the present invention may also be formulated as suspensions in aqueous, non-aqueous, or mixed media. Aqueous suspensions may further contain substances which increase the viscosity of the suspension including, for example, sodium carboxymethylcellulose, sorbitol and/or dextran. The suspension may also contain stabilizers.

The compositions of the present invention may be prepared and formulated as emulsions, or to contain emulsions. Emulsions may contain additional components which may be present as a solution in either the aqueous phase, oily phase, or itself as a separate phase. Pharmaceutical excipients such as emulsifiers, stabilizers, dyes, and anti-oxidants may also be present in emulsions as needed. Pharmaceutical emulsions may also be multiple emulsions that are comprised of more than two phases such as, for example, in the case of oil-in-water-in-oil and water-in-oil-in water emulsions. Emulsions according to the invention include oil-in-water emulsions, water-in-oil emulsions, oil-in-water-in-oil emulsions, and water-in-oil-in-water emulsions. Emulsions may be characterized by little or no thermodynamic stability. Often, the dispersed or discontinuous phase of the emulsion is well dispersed into the external or continuous phase and maintained in this form through the means of emulsifiers or the viscosity of the formulation. Either of the phases of the emulsion may be a semisolid or a solid. Other means of stabilizing emulsions entail the use of emulsifiers that may be incorporated into either phase of the emulsion.

Naturally-occurring emulsifiers used in emulsion formulations include lanolin, beeswax, phosphatides, lecithin and acacia. Absorption bases possess hydrophilic properties such that they can soak up water to form water-in-oil emulsions yet retain their semisolid consistencies, such as anhydrous lanolin and hydrophilic petrolatum. Finely divided solids have also been used as good emulsifiers especially in combination with surfactants and in viscous preparations. These include polar inorganic solids, such as heavy metal hydroxides, non-swelling clays such as bentonite, attapulgite, hectorite, kaolin, montmorillonite, colloidal aluminum silicate and colloidal magnesium aluminum silicate, pigments and nonpolar solids such as carbon or glyceryl tristearate.

Various non-emulsifying materials are also be included in emulsion formulations and contribute to the properties of emulsions. These include fats, oils, waxes, fatty acids, fatty alcohols, fatty esters, humectants, hydrophilic colloids, preservatives, antioxidants, or any combination thereof. Hydrophilic colloids or hydrocolloids include naturally occurring gums and synthetic polymers such as polysaccharides (for example, acacia, agar, alginic acid, carrageenan, guar gum, karaya gum, and tragacanth), cellulose derivatives (for example, carboxymethylc cellulose and carboxypropyl cellulose), and synthetic polymers (for example, carbomers, cellulose ethers, and carboxyvinyl polymers).

Commonly used preservatives included in emulsion formulations include methyl paraben, propyl paraben, quaternary ammonium salts, benzalkonium chloride, esters of p-hydroxybenzoic acid, and boric acid. Antioxidants are also commonly added to emulsion formulations to prevent deterioration of the formulation. Antioxidants used may be free radical scavengers such as tocopherols, alkyl gallates, butylated hydroxyanisole, butylated hydroxytoluene, or reducing agents such as ascorbic acid and sodium metabisulfite, and antioxidant synergists such as citric acid, tartaric acid, and lecithin.

The compositions of the present invention may be prepared and formulated as microemulsions, or to contain microemulsions. Microemulsions may improve drug solubilization, protect the drug from enzymatic hydrolysis, possibly enhance drug absorption due to surfactant-induced alterations in membrane fluidity and permeability, ease of preparation, ease of oral administration over solid dosage forms, improve clinical potency, and decrease toxicity. Often microemulsions may form spontaneously at ambient temperature. Microemulsion compositions and formulations of the present invention may facilitate systemic absorption of the compositions from the gastrointestinal tract, as well as improve the local cellular uptake of delivered exosome preparations within the gastrointestinal tract, vagina, and other areas of non-parenteral administration.

Microemulsions of the present invention may also contain additional components and additives such as sorbitan monostearate (Grill 3), Labrasol, and penetration enhancers to improve the properties of the formulation and to enhance the absorption of the oligonucleotides and nucleic acids of the present invention. Penetration enhancers used in the microemulsions of the present invention may be classified as belonging to one of five broad categories—surfactants, fatty acids, bile salts, chelating agents, and non-chelating non-surfactants.

The compositions of this invention can be converted in a known manner into the customary formulations, such as capsules, coated capsules, tablets, coated tablets, pills, pellets, granules, aerosols, syrups, emulsions, suspensions, gels, lotions, pastes, creams, balms and solutions, or any combination thereof, using inert, non-toxic, pharmaceutically suitable excipients or solvents. The therapeutically active compound should in each case be present in amounts calculated to ensure the desired therapeutic effect. Compositions may be formulated in a conventional manner using additional pharmaceutically acceptable carriers or excipients as appropriate. Thus, the composition may be prepared by conventional means with carriers or excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., starch or sodium starch glycolate); or wetting agents (e.g., sodium lauryl sulfate).

In some embodiments, the composition of the invention may further comprise hydroxypropyl methyl cellulose (HPMC).

In some embodiments, the composition of the invention may further comprise about 0.63% to about 1.5% hydroxypropyl methyl cellulose (HPMC).

In certain embodiments, the pharmaceutical compositions of the invention further comprise a penetration enhancer such as a fatty acid, a bile salt, a chelating agent, a surfactant, and a non-surfactant such as an unsaturated cyclic urea, a 1-alkyl-alkanone, a 1-alkenylazacyclo-alakanone, or a steroidal anti-inflammatory agent.

In some embodiments, the composition is contained in capsules, which may be coated by methods well known in the art. Capsules used for non-parenteral delivery may include formulations that are well known in the art.

In some embodiments, the capsule may comprise a single-piece capsule, two-piece capsule, transparent capsule, non-transparent capsule, opaque capsule, slow-release capsule, extended-release capsule, standard-release capsule, rapid-release capsule, quick-release capsule, hard-shell capsule, soft gel capsule, gel capsule, hard gelatin capsule, soft gelatin capsule, animal-based capsule, vegetarian capsule, polysaccharide capsule, cellulose capsule, mucopolysaccharide capsule, tapioca capsule, hydroxypropylmethyl cellulose (HPMC) capsule, pullulan capsule, enteric capsule, uncoated capsule, coated capsule, capsule comprising titanium dioxide, fatty acids, waxes, shellac, plastics, plasticizers, glycerin, sorbitol, plant fibers, additives, preservatives, colorants, or any combination thereof.

Further, multicompartment capsules, lipid coated capsules, water permeable capsules, capsules with control release properties, capsules with multi-stage drug delivery system, or any combination thereof, may also be used to formulate the compositions of the present invention.

Formulations of the present invention may include sterile and non-sterile aqueous solutions, non-aqueous solutions in common solvents such as alcohols, or solutions of the nucleic acids in liquid or solid oil bases. The solutions may also contain buffers, diluents and other suitable additives. Pharmaceutically acceptable organic or inorganic carrier substances suitable for non-parenteral administration which do not deleteriously react with the components of the composition of mammalian exosomes can also be included in the formulation. Suitable pharmaceutically acceptable carriers include, but are not limited to, water, salt solutions, alcohol, polyethylene glycols, gelatin, lactose, amylose, magnesium stearate, talc, silicic acid, viscous paraffin, hydroxymethylcellulose, polyvinylpyrrolidone and the like. The formulations can be sterilized and, if desired, mixed with auxiliary agents, e.g., lubricants, preservatives, stabilizers, wetting agents, emulsifiers, salts for influencing osmotic pressure, buffers, colorings, flavorings, aromatic substances, or any combination thereof. Aqueous suspensions may contain substances which increase the viscosity of the suspension including, for example, sodium carboxymethylcellulose, sorbitol and/or dextran. The suspension may also contain stabilizers.

In some embodiments, solutions according to the invention consist essentially of the nucleosidic moiety. In some embodiments, the present invention provides pharmaceutical compositions comprising at least one nucleosidic moiety such as a nucleoside, nucleotide, or nucleic acid in a solution or emulsion. The nucleic acid can be a ribozyme, a PNA, or an aptamer, but preferably is an oligonucleotide. In some embodiments, solutions according to the invention consist essentially of the nucleosidic moiety and a solvent comprising, for example, saline solution or cocoa butter.

In some embodiments, solutions according to the invention consist essentially of the nucleosidic moiety and a solvent comprising, for example, saline solution or cocoa butter.

The formulations, which may conveniently be presented in unit dosage form, may be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product.

Non-parenteral administration of the compositions of the present invention may involve suppositories. Suppositories are well known in the art. They are generally formulated to be solid at room temperature with a melting temperature below the normal human body temperature of 36 or 37° C. It is therefore common to formulate suppositories with a fat base, such as cocoa butter, which fulfils the above melting point criteria. In some embodiments, the suppository dosage form may also comprise further excipients such as but not limited to binders and adhesives, lubricants, disintegrants, colorants and bulking agents. In some embodiments, the suppository comprises a combination of any of the above-mentioned base substances.

Non-parenteral administration of the compositions of the present invention may involve coating materials, which may modify the delivery of therapeutic agents. Coating materials may facilitate administration without hampering the pharmacological activity of the product.

In some embodiments, suppositories, containing one or more fatty acids, are coated with fatty or oleaginous bases such as cocoa butter, hard fat and hydrogenated vegetable oil, waxes, water-soluble or water-miscible bases such as polyethylene glycols, glycol-surfactant combinations and polyoxyethylene sorbitan fatty acid esters, and combinations thereof. Furthermore, coated suppositories can be in the form of rectal capsules where a free fatty acid containing core is covered by water-soluble compound such as gelatin.

In some embodiments, the composition may further comprise cocoa butter, coconut oil, glycerinated gelatin, hydrogenated oil, polyethylene glycol (PEG), or a fatty acid ester of PEG.

It may be useful to include in the suppository dosage form of the invention an excipient oil component such as a triacylglyceride oil, to reduce discomfort during action of the medicament and bowel movements, composed accordingly in order to have a desired melting point of the overall composition of the dosage form.

The compositions of the present invention encompass any pharmaceutically acceptable compound which, upon administration to an individual, is capable of providing (directly or indirectly) the biologically active metabolite or residue thereof. Accordingly, for example, the disclosure is also drawn to “prodrugs” and “pharmaceutically acceptable salts” of the compounds of the invention and other bioequivalents.

Also provided are methods for treating an animal comprising administering to the animal a therapeutically effective amount of a pharmaceutical composition according to the invention. The composition can be administered via non-parenteral routes, for example, buccal, sublingual, endoscopic, rectal, oral, vaginal, topical, pulmonary, or urethral routes. In preferred embodiments, the compositions of the invention are administered vaginally or rectally by means of an enema or a suppository.

### Uses

Described herein are systems useful for the non-parenteral oral, rectal or vaginal delivery of exosomes to the alimentary canal, skin, eyes, pulmonary tract, urethra, vagina, or circulatory system of a subject. The systems can be used to deliver exosomes across a barrier and into the blood or lymphatic system by a rectal or vaginal route. The systems described herein can be used to deliver a therapeutic dose of mammalian exosomes to a subject. In certain embodiments, the systems are used to deliver a therapeutic dose of human exosomes to a human subject. In certain embodiments, the systems are used to deliver a therapeutic dose of human exosomes to a human subject diagnosed with, suspected of being afflicted with, or at risk of being afflicted with a disease or disorder.

In certain embodiments, the systems and mammalian exosome compositions deliver a therapeutic dose of exosomes to the colon (e.g. large intestine) of an individual diagnosed with, suspected of being afflicted with, or at risk of being afflicted with a disease or disorder. In certain embodiments, the systems deliver a therapeutic dose of exosomes to the small intestine of an individual diagnosed with, suspected of being afflicted with, or at risk of being afflicted with a disease or disorder. In certain embodiments, the systems deliver a therapeutic dose of exosomes to the vagina of an individual diagnosed with, suspected of being afflicted with, or at risk of being afflicted with a disease or disorder. In certain embodiments, the systems deliver a therapeutic dose of exosomes to the ileum of an individual diagnosed with, suspected of being afflicted with, or at risk of being afflicted with a disease or disorder. In certain embodiments, the systems deliver a therapeutic dose of exosomes to the duodenum of an individual diagnosed with, suspected of being afflicted with, or at risk of being afflicted with a disease or disorder. In certain embodiments, the systems deliver a therapeutic dose of exosomes to the jejunum of an individual diagnosed with, suspected of being afflicted with, or at risk of being afflicted with a disease or disorder. In certain embodiments, the systems deliver a therapeutic dose of exosomes to the vagina of an individual diagnosed with, suspected of being afflicted with, or at risk of being afflicted with a disease or disorder. In certain embodiments, the individual is a human individual and the exosomes are human exosomes.

The non-parenteral, oral, rectal or vaginal systems used herein are useful for delivering a therapeutic dose of mammalian exosomes to domesticated animals for veterinary use. In certain embodiments, the systems deliver a therapeutic dose of canine exosomes to dogs. In certain embodiments, the systems deliver a therapeutic dose of canine exosomes to dogs with inflammatory bowel disease. In certain embodiments, the systems deliver a therapeutic dose of feline exosomes to cats. In certain embodiments, the systems deliver a therapeutic dose of bovine exosomes to bovine species. The rectal or vaginal systems described herein may also be mixed with other treatments rectally or vaginally administered to animals. In certain embodiments, systems containing canine exosomes may be mixed with food to deliver to dogs. In certain embodiments, systems containing feline exosomes may be mixed with food to deliver to cats. In certain embodiments, systems containing bovine exosomes may be mixed with food or nutritive feed to deliver to bovine species.

The delivery systems and compositions of mammalian exosomes are, in certain embodiments, for use in treating an inflammatory or autoimmune disorder in a human. In certain embodiments, the inflammatory or autoimmune disorder is a gastrointestinal inflammatory or autoimmune disorder. In certain embodiments, the gastrointestinal inflammatory or autoimmune disorder comprises inflammatory bowel disease or Crohn's disease. In certain embodiments, the gastrointestinal inflammatory or autoimmune disorder comprises Crohn's disease.

The delivery systems and compositions of mammalian exosomes are, in certain embodiments, for use in treating an inflammatory or autoimmune disorder in an animal. In certain embodiments, the inflammatory or autoimmune disorder is a gastrointestinal inflammatory or autoimmune disorder. In certain embodiments, the animal is any one or more of a dog, a cow, a cat, a pig, a horse, or a sheep.

### Exosomes

The exosomes described herein are derived from mammalian cells, isolated and purified accordingly to methods known in the art.

The non-parenteral, oral, rectal, or vaginal delivery systems and compositions of mammalian exosomes described herein are useful for delivery of exosomes to the alimentary canal of a subject. In certain embodiments, the exosomes comprise eukaryotic exosomes. In certain embodiments, the exosomes comprise eukaryotic exosomes that lack a cell-wall. In certain embodiments, the exosomes comprise mammalian exosomes. In certain embodiments, the exosomes comprise human exosomes. In certain embodiments, the exosomes comprise the exosomes of a domestic animal selected from a cat, dog, pig, sheep, horse, cow, goat, yak, and any combination thereof. The exosomes to be included in the non-parenteral, oral, rectal or vaginal delivery systems are those that can deliver a therapeutic effect and halt, reduce, or ameliorate the symptoms of at least one disease associated with the subject, that the subject has been diagnosed with, or is suspected of being afflicted with. In certain embodiments, the disease is a disease of the digestive tract selected from Crohn's disease or inflammatory bowel disease. In certain embodiments, the exosomes do not comprise prokaryotic exosomes.

Stem cells comprise one type of cell useful for producing exosomes for inclusion in the non-parenteral, oral, rectal, or vaginal delivery systems described herein. In certain embodiments, the rectal or vaginal delivery system comprises a plurality of stem exosomes derived or isolated and purified from stem cells. In certain embodiments, the stem cells comprise embryonic stem cells, pluripotent stem cells, adult stem cells, induced pluripotent stem cells, mesenchymal stem cells, hematopoietic stem cells, myoblasts, fibroblasts, hepatic stem cells, kidney stem cells, heart stem cells, or intestinal stem cells, or mixtures thereof. In certain embodiments, the cells comprise embryonic stem cells. In certain embodiments, the cells comprise mesenchymal stem cells. In certain embodiments, the cells comprise type I (pro-inflammatory) mesenchymal stem cells. In certain embodiments, the cells comprise type II (anti-inflammatory) mesenchymal stem cells. In certain embodiments, the cells comprise pluripotent or induced pluripotent stem cells. Type 1 or Type 2 mesenchymal stem cells can be made using TLR4 or TLR3 agonists respectively. Methods of making Type 1 or Type 2 mesenchymal stem cells are described in US2014/0017787 or WO2016053758A1 which are incorporated by reference herein in their entirety. In certain embodiments, the type 2 MSC express higher levels of CXCL9 than an MSC2 not induced with TLR3.

Immune cells are another type of cell useful for producing exosomes for inclusion in the non-parenteral, oral, rectal or vaginal delivery systems described herein. In certain embodiments, the non-parenteral, oral, rectal, or vaginal delivery system comprises a plurality of immune cells. In certain embodiments, the immune cells comprise B cells (e.g., CD19+ cells), T cells (e.g., CD3+ cells), CD8+ T cells, CD4+ T cells, NK cells, dendritic cells, or macrophage cells. In certain embodiments, the immune cells comprise T cells. In certain embodiments, the T cells comprise T cells with a regulatory T cell phenotype that express and secrete IL-10, express and secrete TGF-β, or that express the transcription factor FoxP3. In certain embodiments, the immune cells comprise NK cells. In certain embodiments, the immune cells comprise macrophages. In certain embodiments, the macrophages comprise M1 type (pro-inflammatory) macrophages. In certain embodiments, the macrophages comprise M2 type (suppressor) macrophages.

The non-parenteral, oral, rectal, or vaginal delivery systems and compositions of mammalian exosomes described herein, comprise in certain embodiments, mixtures of different exosome types (e.g., exosomes from different types of cells). In certain embodiments, the mixture comprises 2, 3, 4, 5, 6, 7, 8, 9, or more different exosome types. In certain embodiments, the mixture comprises stem cell exosomes and immune cell exosomes. In certain embodiments, the mixture comprises different stem cell exosomes. In certain embodiments, the mixture comprises different immune exosome types. In certain embodiments, the mixture comprises stem cell exosomes and immune cell exosomes. The mixture can be in any ratio that is attractive for therapeutic treatment. In certain embodiments, two different cell types are included, and the ratio of a first type to a second type is 1:1, 1:2, 1:3, 1:4, 1:5, 1:6, 1:7, 1:8, or 1:9.

The non-parenteral, oral, rectal, or vaginal delivery systems and compositions of mammalian exosomes described herein comprise exosomes in a therapeutic amount. In certain embodiments, a therapeutic amount of exosomes comprise

### Matrix

The exosomes of the currently described non-parenteral, oral, rectal, or vaginal delivery systems and compositions of mammalian exosomes comprise, or are embedded in a matrix, gel, or excipient. The matrix serves to protect exosomes from dehydration, osmotic stress, pH stress, protease degradation, and other stresses present in the GI system. In certain embodiments, the exosomes are embedded or suspended in a matrix. In certain embodiments, the matrix comprises agar. In certain embodiments, the matrix comprises methyl cellulose. In certain embodiments, the matrix comprises alginate. In certain embodiments, the matrix comprises methyl cellulose and alginate. In certain embodiments, the matrix comprises methyl cellulose and alginate in approximately equal amounts. In certain embodiments, the matrix comprises a hydrogel. Suitable hydrogels include those derived from collagen, hyaluronate, hyaluronan, fibrin, alginate, agarose, chitosan, and combinations thereof. In other embodiments, suitable hydrogels are synthetic polymers. In further embodiments, suitable matrices for embedding exosomes include those derived from poly(acrylic acid) and derivatives thereof, poly(ethylene oxide) and copolymers thereof, poly(vinyl alcohol), polyphosphazene, and combinations thereof. In various specific embodiments, the confinement material is selected from: hydrogel, NovoGel™ agarose, alginate, gelatin, Matrigel™, hyaluronan, poloxamer, peptide hydrogel, poly(isopropyl n-polyacrylamide), polyethylene glycol diacrylate (PEG-DA), hydroxyethyl methacrylate, polydimethylsiloxane, polyacrylamide, poly(lactic acid), or combinations thereof.

In certain embodiments the matrix comprises alginate gel and methyl cellulose in approximately equal amounts.

In certain embodiments, the matrix comprises exosomes mixed with alginate gel and methyl cellulose at a given ratio. In certain embodiments, the ratio of alginate gel to methyl cellulose in the matrix is about 1:0.1, about 1:0.2, about 1:0.3 about 1:0.4, about 1:0.5, about 1:0.6, about 1:0.7, about 1:0.8, about 1:0.9, about 1:1, about 1:1.1, about 1:1.2, about 1:1.3, about 1:1.4, about 1:1.5, about 1:1.6, about 1:1.7, about 1:1.8, about 1:1.9, or about 1:2. In certain embodiments, the ratio of methyl cellulose to alginate gel in the matrix is about 1:0.1, about 1:0.2, about 1:0.3 about 1:0.4, about 1:0.5, about 1:0.6, about 1:0.7, about 1:0.8, about 1:0.9, about 1:1, about 1:1.1, about 1:1.2, about 1:1.3, about 1:1.4, about 1:1.5, about 1:1.6, about 1:1.7, about 1:1.8, about 1:1.9, or about 1:2.

Alternatively, in certain embodiments, this mixture of alginate gel and methyl cellulose may contain different concentrations of methyl cellulose. In certain embodiments, the concentration of alginate gel is about 0.5% to about 5.0%. In certain embodiments, the concentration of alginate gel is about 0.5% to about 4.0%. In certain embodiments, the concentration of alginate gel is about 0.5% to about 3.0%. In certain embodiments, the concentration of alginate gel is at least about 0.5%. In certain embodiments, the concentration of alginate gel is at most about 3.0%. In certain embodiments, the concentration of alginate gel is about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, about 2.0%, about 2.1%, about 2.2%, about 2.3%, about 2.4%, about 2.5%, about 2.6%, about 2.7%, about 2.8%, about 2.9%, or about 3.0%. In certain embodiments, the concentration of alginate gel is at least about 0.5%, at least about 0.6%, at least about 0.7%, at least about 0.8%, at least about 0.9%, at least about 1.0%, at least about 1.1%, at least about 1.2%, at least about 1.3%, or at least about 1.4%. In certain embodiments, the concentration of alginate gel is at most about 3.0% at most about 2.9%, at most about 2.8%, at most about 2.7%, at most about 2.6%, at most about 2.5%, at most about 2.4%, at most about 2.3%, at most about 2.2%, at most about 2.1%, at most about 2.0%, at most about 1.9%, at most about 1.8%, at most about 1.7%, at most about 1.6%, or at most about 1.5%. In certain embodiments, the concentration of alginate gel is about 0.5% to about 0.7%, about 0.5% to about 0.9%, about 0.5% to about 1.1%, about 0.5% to about 1.3%, about 0.5% to about 1.5%, about 0.5% to about 1.7%, about 0.5% to about 1.9%, about 0.5% to about 2.1%, about 0.5% to about 2.5%, about 0.5% to about 2.7%, about 0.5% to about 2.9%, about 0.7% to about 0.9%, about 0.7% to about 1.1%, about 0.7% to about 1.3%, about 0.7% to about 1.5%, about 0.7% to about 1.7%, about 0.7% to about 1.9%, about 0.7% to about 2.1%, about 0.7% to about 2.5%, about 0.7% to about 2.7%, about 0.7% to about 2.9%, about 0.9% to about 1.1%, about 0.9% to about 1.3%, about 0.9% to about 1.5%, about 0.9% to about 1.7%, about 0.9% to about 1.9%, about 0.9% to about 2.1%, about 0.9% to about 2.5%, about 0.9% to about 2.7%, about 0.9% to about 2.9%, about 1.1% to about 1.3%, about 1.1% to about 1.5%, about 1.1% to about 1.7%, about 1.1% to about 1.9%, about 1.1% to about 2.1%, about 1.1% to about 2.5%, about 1.1% to about 2.7%, about 1.1% to about 2.9%, about 1.3% to about 1.5%, about 1.3% to about 1.7%, about 1.3% to about 1.9%, about 1.3% to about 2.1%, about 1.3% to about 2.5%, about 1.3% to about 2.7%, about 1.3% to about 2.9%, about 1.5% to about 1.7%, about 1.5% to about 1.9%, about 1.5% to about 2.1%, about 1.5% to about 2.5%, about 1.5% to about 2.7%, about 1.5% to about 2.9%, about 1.7% to about 1.9%, about 1.7% to about 2.1%, about 1.7% to about 2.5%, about 1.7% to about 2.7%, about 1.7% to about 2.9%, about 1.9% to about 2.1%, about 1.9% to about 2.5%, about 1.9% to about 2.7%, about 1.9% to about 2.9%, about 2.1% to about 2.5%, about 2.1% to about 2.7%, about 2.1% to about 2.9%, about 2.5% to about 2.7%, about 2.5% to about 2.9%, or about 2.7% to about 2.9%.

In certain embodiments, the concentration of alginate gel is about 0.1% to about 1%. In certain embodiments, the concentration of alginate gel is about 0.1% to about 0.25%, about 0.1% to about 0.33%, about 0.1% to about 0.5%, about 0.1% to about 0.67%, about 0.1% to about 0.75%, about 0.1% to about 0.8%, about 0.1% to about 0.9%, about 0.1% to about 1%, about 0.25% to about 0.33%, about 0.25% to about 0.5%, about 0.25% to about 0.67%, about 0.25% to about 0.75%, about 0.25% to about 0.8%, about 0.25% to about 0.9%, about 0.25% to about 1%, about 0.33% to about 0.5%, about 0.33% to about 0.67%, about 0.33% to about 0.75%, about 0.33% to about 0.8%, about 0.33% to about 0.9%, about 0.33% to about 1%, about 0.5% to about 0.67%, about 0.5% to about 0.75%, about 0.5% to about 0.8%, about 0.5% to about 0.9%, about 0.5% to about 1%, about 0.67% to about 0.75%, about 0.67% to about 0.8%, about 0.67% to about 0.9%, about 0.67% to about 1%, about 0.75% to about 0.8%, about 0.75% to about 0.9%, about 0.75% to about 1%, about 0.8% to about 0.9%, about 0.8% to about 1%, or about 0.9% to about 1%. In certain embodiments, the concentration of alginate gel is about 0.1%, about 0.25%, about 0.33%, about 0.5%, about 0.67%, about 0.75%, about 0.8%, about 0.9%, or about 1%. In certain embodiments, the concentration of alginate gel is at least about 0.1%, about 0.25%, about 0.33%, about 0.5%, about 0.67%, about 0.75%, about 0.8%, or about 0.9%. In certain embodiments, the concentration of alginate gel is at most about 0.25%, about 0.33%, about 0.5%, about 0.67%, about 0.75%, about 0.8%, about 0.9%, or about 1%.

In certain embodiments, the concentration of methyl cellulose is about 0.5% to about 3.0%. In certain embodiments, the concentration of methyl cellulose is at least about 0.5%. In certain embodiments, the concentration of methyl cellulose is at most about 3.0%. In certain embodiments, the concentration of methyl cellulose is about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, about 2.0%, about 2.1%, about 2.2%, about 2.3%, about 2.4%, about 2.5%, about 2.6%, about 2.7%, about 2.8%, about 2.9%, or about 3.0%. In certain embodiments, the concentration of methyl cellulose is at least about 0.5%, at least about 0.6%, at least about 0.7%, at least about 0.8%, at least about 0.9%, at least about 1.0%, at least about 1.1%, at least about 1.2%, at least about 1.3%, or at least about 1.4%. In certain embodiments, the concentration of methyl cellulose is at most about 3.0% at most about 2.9%, at most about 2.8%, at most about 2.7%, at most about 2.6%, at most about 2.5%, at most about 2.4%, at most about 2.3%, at most about 2.2%, at most about 2.1%, at most about 2.0%, at most about 1.9%, at most about 1.8%, at most about 1.7%, at most about 1.6%, or at most about 1.5%. In certain embodiments, the concentration of methyl cellulose is about 0.5% to about 0.7%, about 0.5% to about 0.9%, about 0.5% to about 1.1%, about 0.5% to about 1.3%, about 0.5% to about 1.5%, about 0.5% to about 1.7%, about 0.5% to about 1.9%, about 0.5% to about 2.1%, about 0.5% to about 2.5%, about 0.5% to about 2.7%, about 0.5% to about 2.9%, about 0.7% to about 0.9%, about 0.7% to about 1.1%, about 0.7% to about 1.3%, about 0.7% to about 1.5%, about 0.7% to about 1.7%, about 0.7% to about 1.9%, about 0.7% to about 2.1%, about 0.7% to about 2.5%, about 0.7% to about 2.7%, about 0.7% to about 2.9%, about 0.9% to about 1.1%, about 0.9% to about 1.3%, about 0.9% to about 1.5%, about 0.9% to about 1.7%, about 0.9% to about 1.9%, about 0.9% to about 2.1%, about 0.9% to about 2.5%, about 0.9% to about 2.7%, about 0.9% to about 2.9%, about 1.1% to about 1.3%, about 1.1% to about 1.5%, about 1.1% to about 1.7%, about 1.1% to about 1.9%, about 1.1% to about 2.1%, about 1.1% to about 2.5%, about 1.1% to about 2.7%, about 1.1% to about 2.9%, about 1.3% to about 1.5%, about 1.3% to about 1.7%, about 1.3% to about 1.9%, about 1.3% to about 2.1%, about 1.3% to about 2.5%, about 1.3% to about 2.7%, about 1.3% to about 2.9%, about 1.5% to about 1.7%, about 1.5% to about 1.9%, about 1.5% to about 2.1%, about 1.5% to about 2.5%, about 1.5% to about 2.7%, about 1.5% to about 2.9%, about 1.7% to about 1.9%, about 1.7% to about 2.1%, about 1.7% to about 2.5%, about 1.7% to about 2.7%, about 1.7% to about 2.9%, about 1.9% to about 2.1%, about 1.9% to about 2.5%, about 1.9% to about 2.7%, about 1.9% to about 2.9%, about 2.1% to about 2.5%, about 2.1% to about 2.7%, about 2.1% to about 2.9%, about 2.5% to about 2.7%, about 2.5% to about 2.9%, or about 2.7% to about 2.9%.

In certain embodiments, the concentration of methyl cellulose 1 is about 0.1% to about 1%. In certain embodiments, the concentration of methyl cellulose 1 is about 0.1% to about 0.25%, about 0.1% to about 0.33%, about 0.1% to about 0.5%, about 0.1% to about 0.67%, about 0.1% to about 0.75%, about 0.1% to about 0.8%, about 0.1% to about 0.9%, about 0.1% to about 1%, about 0.25% to about 0.33%, about 0.25% to about 0.5%, about 0.25% to about 0.67%, about 0.25% to about 0.75%, about 0.25% to about 0.8%, about 0.25% to about 0.9%, about 0.25% to about 1%, about 0.33% to about 0.5%, about 0.33% to about 0.67%, about 0.33% to about 0.75%, about 0.33% to about 0.8%, about 0.33% to about 0.9%, about 0.33% to about 1%, about 0.5% to about 0.67%, about 0.5% to about 0.75%, about 0.5% to about 0.8%, about 0.5% to about 0.9%, about 0.5% to about 1%, about 0.67% to about 0.75%, about 0.67% to about 0.8%, about 0.67% to about 0.9%, about 0.67% to about 1%, about 0.75% to about 0.8%, about 0.75% to about 0.9%, about 0.75% to about 1%, about 0.8% to about 0.9%, about 0.8% to about 1%, or about 0.9% to about 1%. In certain embodiments, the concentration of methyl cellulose 1 is about 0.1%, about 0.25%, about 0.33%, about 0.5%, about 0.67%, about 0.75%, about 0.8%, about 0.9%, or about 1%. In certain embodiments, the concentration of methyl cellulose 1 is at least about 0.1%, about 0.25%, about 0.33%, about 0.5%, about 0.67%, about 0.75%, about 0.8%, or about 0.9%. In certain embodiments, the concentration of methyl cellulose 1 is at most about 0.25%, about 0.33%, about 0.5%, about 0.67%, about 0.75%, about 0.8%, about 0.9%, or about 1%.

In some embodiments, the matrix comprises exosomes mixed with a matrix of about 0.5% to about 3.0% alginate gel and about 0.5% to about 3.0% methyl cellulose. The cell gel mixture is then transferred to a lipophilic or hydrophilic-coated capsule. In certain embodiments, the capsule is coated with methyl methacrylate-methacrylic acid copolymer (1:1). In certain embodiments, the mammalian exosomes comprise mesenchymal stem cell exosomes.

In some embodiments, the matrix comprises mammalian exosomes mixed with a matrix of about 1.5% alginate gel and about 1.5% methyl cellulose. The exosome gel mixture is then transferred to a lipophilic or hydrophilic-coated capsule. In certain embodiments, the capsule is coated with methyl methacrylate-methacrylic acid copolymer (1:1). In certain embodiments, the mammalian exosomes comprise mesenchymal stem cell exosomes.

In some embodiments, the matrix comprises mammalian exosomes suspended in mixture of about 0.5% to about 3.0% alginate gel and about 0.5% to about 3.0% methyl cellulose, in a capsule coated with methyl methacrylate-methacrylic acid copolymer (1:1). In certain embodiments, the mammalian exosomes comprise mesenchymal stem cell exosomes.

In some embodiments, the matrix comprises mammalian exosomes suspended in mixture of about 1.0% to about 2.5% alginate gel and about 1.0% to about 2.5% methyl cellulose, in a capsule coated with methyl methacrylate-methacrylic acid copolymer (1:1). In certain embodiments, the mammalian exosomes comprise mesenchymal stem cell exosomes.

In some embodiments, the matrix comprises mammalian exosomes suspended in mixture of about 1.0% to about 2.0% alginate gel and about 1.0% to about 2.0% methyl cellulose, in a capsule coated with methyl methacrylate-methacrylic acid copolymer (1:1). In certain embodiments, the mammalian exosomes comprise mesenchymal stem cell exosomes.

In some embodiments, the matrix comprises mammalian exosomes suspended in mixture of about 1.2% to about 1.8% alginate gel and about 1.2% to about 1.8% methyl cellulose, in a capsule coated with methyl methacrylate-methacrylic acid copolymer (1:1).

In some embodiments, the matrix comprises mammalian exosomes suspended in mixture of about 1.4% to about 1.6% alginate gel and about 1.4% to about 1.6% methyl cellulose, in a capsule coated with methyl methacrylate-methacrylic acid copolymer (1:1). In certain embodiments, the mammalian exosomes comprise mesenchymal stem cell exosomes.

In some embodiments, the matrix comprises mammalian exosomes suspended in mixture of about 0.25% to about 0.75% alginate gel and about 0.25% to about 0.75% methyl cellulose, in a capsule coated with methyl methacrylate-methacrylic acid copolymer (1:1). In certain embodiments, the mammalian exosomes comprise mesenchymal stem cell exosomes.

In some embodiments, the matrix comprises mammalian exosomes suspended in mixture of about 0.1% to about 0.9% alginate gel and about 0.1% to about 0.9% methyl cellulose, in a capsule coated with methyl methacrylate-methacrylic acid copolymer (1:1). In certain embodiments, the mammalian exosomes comprise mesenchymal stem cell exosomes.

In some embodiments, the matrix comprises mammalian exosomes suspended in mixture of about 1.5% alginate gel and about 1.5% methyl cellulose, in a capsule coated with methyl methacrylate-methacrylic acid copolymer (1:1). In certain embodiments, the mammalian exosomes comprise mesenchymal stem cell exosomes.

In certain embodiments, the matrix comprises exosomes and agar at a given concentration. In certain embodiments, the concentration of agar in the matrix is between about 0.1% w/v and about 10% w/v, between about 0.1% w/v and about 8% w/v, between about 0.1% w/v and about 5% w/v, between about 0.1% w/v and about 4% w/v, between about 0.1% w/v and about 3% w/v, between about 0.1% w/v and about 2% w/v, between about 0.1% w/v and about 1% w/v, between about 0.2% w/v and about 10% w/v, between about 0.2% w/v and about 8% w/v, between about 0.2% w/v and about 5% w/v, between about 0.2% w/v and about 4% w/v, between about 0.2% w/v and about 3% w/v, between about 0.2% w/v and about 2% w/v, between about 0.2% w/v and about 1% w/v, between about 0.4% w/v and about 10% w/v, between about 0.4% w/v and about 8% w/v, between about 0.4% w/v and about 5% w/v, between about 0.4% w/v and about 4% w/v, between about 0.4% w/v and about 3% w/v, between about 0.4% w/v and about 2% w/v, between about 0.4% w/v and about 1% w/v, between about 0.5% w/v and about 10% w/v, between about 0.5% w/v and about 8% w/v, between about 0.5% w/v and about 5% w/v, between about 0.5% w/v and about 4% w/v, between about 0.5% w/v and about 3% w/v, between about 0.5% w/v and about 2% w/v, between about 0.5% w/v and about 1% w/v. In certain embodiments, the concentration of Agar in the matrix is about 0.1%, 0.2%, 0.3%, 0.4%, 0.5%, 0.6%, 0.7%, 0.8%, 0.9%, or 1.0%. In certain embodiments, the concentration of Agar in the matrix is about 1.0%, 2.0%, 3.0%, 4.0%, 5.0%, 6.0%, 7.0%, 8.0%, 9.0%, or 10%.

The matrixes described herein have a given viscosity. In further embodiments, the matrix comprising the exosomes is characterized by having a viscosity of between about 500 and 1,000,000 centipoise; between about 1000 and 1,000,000 centipoise; between about 1,000 and 500,000 centipoise; between about 10,000 and 500,000 centipoise; between about 10,000 and 400,000 centipoise; between about 10,000 and 3000,000 centipoise; between about 100,000 and 1,000,000 centipoise; or between about 500,000 and 1,000,000 centipoise.

In certain embodiments, the matrix comprises additional molecules such as an extracellular matrix protein, like collagen or fibrin; a carbohydrate, such as glucose, dextrose, or sucrose; fibers, such as cellulose; a vitamin or mineral; buffers, isotonic solutions, or amino acids.

The exosomes of the current disclosure can be characterized in a variety of ways. In certain embodiments, the exosomes comprise a protein or polypeptide selected from the list consisting of CD81, CD63, CD9, HSP60, HSP70, HSP90, VEGF, PGE2, and combinations thereof. The exosome particles may be characterized by standard method known in the art such as flow cytometry with specific antibody as compared to an isotype control antibody. In certain embodiments, the exomes may be characterized by a 2-fold, 3-fold, 4-fold, 5-fold, 6-fold, 7-fold, 8-fold, 9-fold, 10-fold or greater increase in CD81 protein compared to an isotype control antibody. In certain embodiments, the exomes may be characterized by a 2-fold, 3-fold, 4-fold, 5-fold, 6-fold, 7-fold, 8-fold, 9-fold, 10-fold or greater increase in CD63 protein compared to an isotype control antibody. In certain embodiments, the exomes may be characterized by a 2-fold, 3-fold, 4-fold, 5-fold, 6-fold, 7-fold, 8-fold, 9-fold, 10-fold or greater increase in CD9 protein compared to an isotype control antibody. In certain embodiments, the exomes may be characterized by a 2-fold, 3-fold, 4-fold, 5-fold, 6-fold, 7-fold, 8-fold, 9-fold, 10-fold or greater increase in HSP60 protein compared to an isotype control antibody. In certain embodiments, the exomes may be characterized by a 2-fold, 3-fold, 4-fold, 5-fold, 6-fold, 7-fold, 8-fold, 9-fold, 10-fold or greater increase in HSP70 protein compared to an isotype control antibody. In certain embodiments, the exomes may be characterized by a 2-fold, 3-fold, 4-fold, 5-fold, 6-fold, 7-fold, 8-fold, 9-fold, 10-fold or greater increase in HHSP90 protein compared to an isotype control antibody. In certain embodiments, the exomes may be characterized by a 2-fold, 3-fold, 4-fold, 5-fold, 6-fold, 7-fold, 8-fold, 9-fold, 10-fold or greater increase in VEGF protein compared to an isotype control antibody. In certain embodiments, the exomes may be characterized by a 2-fold, 3-fold, 4-fold, 5-fold, 6-fold, 7-fold, 8-fold, 9-fold, 10-fold or greater increase in PGE2 protein compared to an isotype control antibody.

The compositions described herein also allow greater number of exosomes to be retrieved after incubation for of the composition at 2 hours at pH 1.2. In certain embodiments, greater than 30%, 40%, 50%, 60%, 70%, 80%, 90% or more intact exosomes can be recovered after a 2 hour incubation at pH 1.2. In certain embodiments, greater than 30% or more intact exosomes can be recovered after a 2 hour incubation at pH 1.2. In certain embodiments, greater than 40% or more intact exosomes can be recovered after a 2 hour incubation at pH 1.2. In certain embodiments, greater than 50% or more intact exosomes can be recovered after a 2 hour incubation at pH 1.2. In certain embodiments, greater than 60% or more intact exosomes can be recovered after a 2 hour incubation at pH 1.2. Intact exosomes can be counted and verified by microscopy.

### Coatings

The oral delivery systems herein comprise a coating formulated to dissolve at a certain pH. For biphasic delivery the oral delivery systems comprise two coatings formulated to dissolve at different pHs. In certain embodiments, the coatings are formulated to dissolve at a pH of about 5.5 to about 6.0 in order to target the duodenum; at a pH of about 7.3 to about 8.0 to target the ileum; or a pH of about 5.5 to about 6.5 to target the colon. In certain embodiments, the coating is formulated to dissolve at a pH of about 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, or 8.0. In certain embodiments, for biphasic delivery one coating is formulated at a pH of about 7.3 to about 8.0 to target the ileum; and one coating at a pH of about 5.5 to about 6.5 to target the colon. In certain embodiments, for biphasic delivery one coating is formulated at a pH of about 5.5 to about 6.0 to target the duodenum; and one coating at a pH of about 5.5 to about 6.5 to target the colon. In certain embodiments, for biphasic delivery one coating is formulated at a pH of about 5.5 to about 6.0 to target the duodenum; and one coating at a pH of about 7.3 to about 8.0 to target the ileum.

Also contemplated are enteric exosomal formulations including a exosome or preparation thereof and an enteric material; and a pharmaceutically acceptable carrier or excipient thereof. Enteric materials refer to polymers that are substantially insoluble in the acidic environment of the stomach, and that are predominantly soluble in intestinal fluids at specific pHs. The small intestine is the part of the gastrointestinal tract (gut) between the stomach and the large intestine, and includes the duodenum, jejunum, and ileum. The pH of the duodenum is about 5.5, the pH of the jejunum is about 6.5 and the pH of the distal ileum is about 7.5. Accordingly, enteric materials are not soluble, for example, until a pH of about 5.0, of about 5.2, of about 5.4, of about 5.6, of about 5.8, of about 6.0, of about 6.2, of about 6.4, of about 6.6, of about 6.8, of about 7.0, of about 7.2, of about 7.4, of about 7.6, of about 7.8, of about 8.0, of about 8.2, of about 8.4, of about 8.6, of about 8.8, of about 9.0, of about 9.2, of about 9.4, of about 9.6, of about 9.8, or of about 10.0. Exemplary enteric materials that can be included in the oral delivery systems and compositions of mammalian exosomes described herein include cellulose acetate phthalate (CAP), hydroxypropyl methylcellulose phthalate (HPMCP), polyvinyl acetate phthalate (PVAP), hydroxypropyl methylcellulose acetate succinate (HPMCAS), cellulose acetate trimellitate, hydroxypropyl methylcellulose succinate, cellulose acetate succinate, cellulose acetate hexahydrophthalate, cellulose propionate phthalate, cellulose acetate maleate, cellulose acetate butyrate, cellulose acetate propionate, copolymer of methylmethacrylic acid and methyl methacrylate, copolymer of methyl acrylate, methylmethacrylate and methacrylic acid, copolymer of methylvinyl ether and maleic anhydride (Gantrez ES series), ethyl methyacrylate-methylmethacrylate-chlorotrimethylammonium ethyl acrylate copolymer, natural resins such as zein, shellac and copal collophorium, and several commercially available enteric dispersion systems (e.g., Eudragit L30D55, Eudragit FS30D, Eudragit L100, Eudragit S100, Kollicoat EMM30D, Estacryl 30D, Coateric, and Aquateric). The solubility of each of the above materials is either known or is readily determinable in vitro.

Biphasic capsules comprising mammalian exosomes comprise a first and a second enteric coating. In certain embodiments, the first enteric coating comprises copolymers of methacrylic acid, methyl methacrylate, cellulose acetate phthalate, cellulose acetate succinate, hydroxypropyl methyl cellulose phthalate, hydroxypropyl methyl cellulose acetate succinate, polyvinyl acetate phthalate, shellac, cellulose acetate trimellitate, sodium alginate, or zein. In certain embodiments, the second enteric coating comprises copolymers of methacrylic acid, methyl methacrylate, cellulose acetate phthalate, cellulose acetate succinate, hydroxypropyl methyl cellulose phthalate, hydroxypropyl methyl cellulose acetate succinate, polyvinyl acetate phthalate, shellac, cellulose acetate trimellitate, sodium alginate, or zein. In certain embodiments, the first enteric coating comprises copolymers of methacrylic acid. In certain embodiments, the second enteric coating comprises copolymers of methacrylic acid.

In certain embodiments, an enteric coating of the mammalian exosomes compositions described herein comprise methyl methacrylate-methacrylic acid copolymer (1:1).

In certain embodiments, the gel, matrix, or excipient along with an enteric coating preserves the viability of the first plurality of mammalian exosomes, and/or the second plurality of mammalian exosomes at about 80-85% viability. In certain embodiments the gel, matrix or excipient preserves the viability of the first plurality of mammalian exosomes, and/or the second plurality of exosomes above about 50%, 60%, 70%, or 80% viability for at least 2, 3, 4, 5, 6, 7, 8, 9, 10 days at 24° C.

In certain embodiments, the gel, matrix along with an enteric coating or excipient provides a Tₘₐₓ of about 0.4 to 0.6 hours. In certain embodiments, the gel, matrix or excipient provides a Tₘₐₓ of about 0.4 hours, about 0.41 hours, about 0.42 hours, about 0.43 hours, about 0.44 hours, about 0.45 hours, about 0.46 hours, about 0.47 hours, about 0.48 hours, about 0.49 hours, about 0.50 hours, about 0.51 hours, about 0.52 hours, about 0.53 hours, about 0.54 hours, about 0.55 hours, about 0.56 hours, about 0.57 hours, about 0.58 hours, about 0.59 hours, or about 0.60 hours. In certain embodiments, the gel, matrix or excipient provides a Tₘₐₓ of at least about 0.4 hours, at least about 0.4 hours, at least about 0.42 hours, at least about 0.43 hours, at least about 0.44 hours, or at least about 0.45 hours. In certain embodiments, the gel, matrix or excipient provides a Tₘₐₓ of at most about 0.46 hours, at most about 0.47 hours, at most about 0.48 hours, at most about 0.49 hours, at most about 0.50 hours, at most about 0.51 hours, at most about 0.52 hours, at most about 0.53 hours, at most about 0.54 hours, at most about 0.55 hours, at most about 0.56 hours, at most about 0.57 hours, at most about 0.58 hours, at most about 0.59 hours, or at most about 0.60 hours.

The non-parenteral, oral, rectal, or vaginal delivery systems herein comprise a coating formulated to dissolve at a certain pH. For biphasic delivery the rectal or vaginal delivery systems comprise two coatings formulated to dissolve at different pHs. In certain embodiments, the coatings are formulated to dissolve at a pH of about 5.5 to about 6.0 in order to target the duodenum; at a pH of about 7.3 to about 8.0 to target the ileum; or a pH of about 5.5 to about 6.5 to target the colon. In certain embodiments, the coating is formulated to dissolve at a pH of about 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, or 8.0. In certain embodiments, for biphasic delivery one coating is formulated at a pH of about 7.3 to about 8.0 to target the ileum; and one coating at a pH of about 5.5 to about 6.5 to target the colon. In certain embodiments, for biphasic delivery one coating is formulated at a pH of about 5.5 to about 6.0 to target the duodenum; and one coating at a pH of about 5.5 to about 6.5 to target the colon. In certain embodiments, for biphasic delivery one coating is formulated at a pH of about 5.5 to about 6.0 to target the duodenum; and one coating at a pH of about 7.3 to about 8.0 to target the ileum.

Also contemplated are enteric formulations including a disclosed exosome or preparation of exosomes and a lipophilic or hydrophilic material; and a pharmaceutically acceptable carrier or excipient thereof. Enteric materials refer to polymers that are substantially insoluble in the acidic environment of the stomach, and that are predominantly soluble in intestinal fluids at specific pHs. The small intestine is the part of the gastrointestinal tract (gut) between the stomach and the large intestine, and includes the duodenum, jejunum, and ileum. The pH of the duodenum is about 5.5, the pH of the jejunum is about 6.5 and the pH of the distal ileum is about 7.5. Accordingly, enteric materials are not soluble, for example, until a pH of about 5.0, of about 5.2, of about 5.4, of about 5.6, of about 5.8, of about 6.0, of about 6.2, of about 6.4, of about 6.6, of about 6.8, of about 7.0, of about 7.2, of about 7.4, of about 7.6, of about 7.8, of about 8.0, of about 8.2, of about 8.4, of about 8.6, of about 8.8, of about 9.0, of about 9.2, of about 9.4, of about 9.6, of about 9.8, or of about 10.0. Exemplary enteric materials that can be included in the rectal or vaginal delivery systems and compositions of mammalian exosomes described herein include cellulose acetate phthalate (CAP), hydroxypropyl methylcellulose phthalate (HPMCP), polyvinyl acetate phthalate (PVAP), hydroxypropyl methylcellulose acetate succinate (HPMCAS), cellulose acetate trimellitate, hydroxypropyl methylcellulose succinate, cellulose acetate succinate, cellulose acetate hexahydrophthalate, cellulose propionate phthalate, cellulose acetate maleate, cellulose acetate butyrate, cellulose acetate propionate, copolymer of methylmethacrylic acid and methyl methacrylate, copolymer of methyl acrylate, methylmethacrylate and methacrylic acid, copolymer of methylvinyl ether and maleic anhydride (Gantrez ES series), ethyl methyacrylate-methylmethacrylate-chlorotrimethylammonium ethyl acrylate copolymer, natural resins such as zein, shellac and copal collophorium, and several commercially available enteric dispersion systems (e.g., Eudragit L30D55, Eudragit FS30D, Eudragit L100, Eudragit S100, Kollicoat EMM30D, Estacryl 30D, Coateric, and Aquateric). The solubility of each of the above materials is either known or is readily determinable in vitro.

Biphasic capsules comprising mammalian exosomes comprise a first and a second enteric coating. In certain embodiments, the first enteric coating comprises copolymers of methacrylic acid, methyl methacrylate, cellulose acetate phthalate, cellulose acetate succinate, hydroxypropyl methyl cellulose phthalate, hydroxypropyl methyl cellulose acetate succinate, polyvinyl acetate phthalate, shellac, cellulose acetate trimellitate, sodium alginate, or zein. In certain embodiments, the second enteric coating comprises copolymers of methacrylic acid, methyl methacrylate, cellulose acetate phthalate, cellulose acetate succinate, hydroxypropyl methyl cellulose phthalate, hydroxypropyl methyl cellulose acetate succinate, polyvinyl acetate phthalate, shellac, cellulose acetate trimellitate, sodium alginate, or zein. In certain embodiments, the first enteric coating comprises copolymers of methacrylic acid. In certain embodiments, the second enteric coating comprises copolymers of methacrylic acid.

In certain embodiments, an enteric coating of the mammalian exosome compositions described herein comprise methyl methacrylate-methacrylic acid copolymer (1:1).

In certain embodiments, the gel, matrix, or excipient along with an enteric coating preserves the stability and potency of mammalian exosomes, and/or the second plurality of mammalian exosomes at about 80-85% potency. In certain embodiments the gel, matrix or excipient preserves the potency of the first plurality of mammalian exosomes, and/or the second plurality of mammalian exosomes above about 50%, 60%, 70%, or 80% potency for at least 2, 3, 4, 5, 6, 7, 8, 9, 10 days at 24° C. Potency or stability can be measured by any method suitable to determine the integrity of the membrane structure of the exosome or the potency and stability (e.g., lack of degradation) of proteins or nucleic acids within the exosomes.

The rectal or vaginal delivery systems herein comprise a coating or coatings formulated to dissolve at a normal body temperature, or normal human body temperature. For biphasic or monophasic delivery the rectal or vaginal delivery systems comprise a coating or coatings formulated to dissolve at different body temperatures. In certain embodiments, the coating or coatings are formulated to dissolve at temperatures of about 36° C. or above. In certain embodiments, the coating or coatings are formulated to dissolve at temperatures of about 36° C. to about 47° C. In certain embodiments, the coating or coatings are formulated to dissolve at temperatures of about 32° C. to about 41° C. In certain embodiments, the coating or coatings are formulated to dissolve at temperatures of about 34° C. to about 45° C. In some embodiments, the coating or coatings can be formulated to dissolve at temperatures of at least about 32° C., 33° C., 34° C., 35° C., 36° C., 37° C., 38° C., 39° C., 40° C., 41° C., 42° C., 43° C., 44° C., 45° C., 46° C., or 47° C. In some embodiments, the coating or coatings can be formulated to dissolve at temperatures of about 32° C., 33° C., 34° C., 35° C., 36° C., 37° C., 38° C., 39° C., 40° C., 41° C., 42° C., 43° C., 44° C., 45° C., 46° C., or 47° C. In some embodiments, the coating or coatings can be formulated to dissolve at temperatures of at most about 32° C., 33° C., 34° C., 35° C., 36° C., 37° C., 38° C., 39° C., 40° C., 41° C., 42° C., 43° C., 44° C., 45° C., 46° C., or 47° C.

Also contemplated are exosome formulations including a disclosed exosome or preparation of exosomes and a lipophilic or hydrophilic material; and a pharmaceutically acceptable carrier or excipient thereof. Lipophilic or hydrophilic coating materials formulated to dissolve at a normal body temperature refer to materials that are substantially insoluble at temperatures that are lower that a normal body temperature, and that are predominantly soluble at temperatures that are at or higher than a normal body temperature. Accordingly, lipophilic or hydrophilic coating materials formulated to dissolve at a normal body temperature are not dissolvable, for example, until a temperature of about 32° C., of about 33° C., of about 34° C., of about 35° C., of about 36° C., of about 37° C., of about 38° C., of about 39° C., of about 40° C., of about 41° C., of about 42° C., of about 43° C., of about 44° C., of about 45° C., of about 46° C., or of about 47° C.

The rectal or vaginal delivery systems herein comprise a coating or coatings formulated to dissolve when in contact with an aqueous solution. For biphasic delivery the rectal or vaginal delivery systems comprise a coating or coatings formulated to dissolve at different aqueous solution volumes. In certain embodiments, the coating or coatings are formulated to dissolve at aqueous solution volumes of about 5.6 mL or above. In certain embodiments, the coating or coatings are formulated to dissolve aqueous solution volumes of about 5.5 mL to about 20.5 mL. In certain embodiments, the coating or coatings are formulated to dissolve at aqueous solution volumes of about 5.5 mL to about 13 mL. In certain embodiments, the coating or coatings are formulated to dissolve at aqueous solution volumes of about 10.5 mL to about 20.5 mL. In some embodiments, the coating or coatings can be formulated to dissolve at aqueous solution volumes of at least about 5.5 mL, 8 mL, 10.5 mL, 13 mL, 15.5 mL, 18 mL, or 20.5 mL. In some embodiments, the coating or coatings can be formulated to dissolve at aqueous solution volumes of about 5.5 mL, 8 mL, 10.5 mL, 13 mL, 15.5 mL, 18 mL, or 20.5 mL. In some embodiments, the coating or coatings can be formulated to dissolve at aqueous solution volumes of at most about 5.5 mL, 8 mL, 10.5 mL, 13 mL, 15.5 mL, 18 mL, or 20.5 mL.

Also contemplated are exosome formulations including a disclosed exosome or preparation of exosomes and a lipophilic or hydrophilic material; and a pharmaceutically acceptable carrier or excipient thereof. Lipophilic or hydrophilic coating materials formulated to dissolve at different aqueous solution volumes refer to materials that are substantially insoluble at volumes that are lower that a normal body fluid volume, and that are predominantly soluble at volumes that are at or higher than a normal body fluid volume. Accordingly, lipophilic or hydrophilic coating materials formulated to dissolve at different aqueous solution volumes are not dissolvable, for example, until a volume of about 5.5 mL, of about 8 mL, of about 10.5 mL, of about 13 mL, of about 15.5 mL, of about 18 mL, or of about 20.5 mL.

Non-parenteral administration of the rectal or vaginal delivery systems herein comprise suppositories and enemas. Suppositories generally formulated to be solid at room temperature and up to at least about 30° C. but having a melting temperature below the normal human body temperature of about 37° C. Suppository formulations in the present disclosure can have melting points that are about 30° C., 31° C., 32° C., 33° C., 34° C., 35° C., 36° C., 37° C., 38° C., 39° C., 40° C., 41° C., 42° C., 43° C., 44° C., 45° C., 46° C., 47° C. or above. It is therefore common to formulate suppositories with a fat base, such as cocoa butter, which fulfils the above melting point criteria. Cocoa butter is a mixture of triglycerides of saturated and unsaturated fatty acids which can be manipulated in solid form at room temperature but melts completely at body temperature. Other fat bases common to formulate suppositories with a fat base include coconut oil, glycerated gelatin, hydrogenated oil, polyethylene glycol (PEG), or a fatty acid ester of PEG. Other materials used to formulate suppositories include hard fat, macrogols, and various gelatinous mixtures consisting of, for example, gelatin, water, and glycerol. Commercially available fat bases suitable for suppository formulations include the above mentioned and in particular Suppocire™ (Gattefosse) lipophilic bases, a semi-synthetic vegetable based oil base available in several grades including Suppocire™ AS, AS2X, NA; Novata™ (Henkel Int.) including Novata A, Novata B, and Novata BC; Witepsol™ (Dynamit Nobel Ab) such as Witepsol™ H5, H12, H15, H32, H35, W25, W31, W32, W32, W35, and W45; Massa Estarinum™ (SASOL), including Massa Estarinum™ of the grades B, BC,E and 299; Japocire™ and Ovucire™. The suppositories of the present invention may suitably comprise any of the above-mentioned materials as base. Hydrophilic waxes can also be used in the invention, such as the polyethylene glycols (e.g. PEG 1500, PEG 3000, PEG 4000 and mixtures thereof). Suppocire AP, is an amphiphilic base comprising saturated polyglycolyzed glycerides. In some embodiments, the suppository dosage form may also comprise further excipients such as but not limited to binders and adhesives, lubricants, disintegrants, colorants and bulking agents. In some embodiments, the suppository comprises a combination of any of the above-mentioned base substances.

Non-parenteral administration of the rectal or vaginal delivery systems herein may optionally comprise coating materials, which may modify the delivery of therapeutic agents. Coating materials may facilitate administration without hampering the therapeutic activity of the product.

In some embodiments, suppositories, containing one or more fatty acids, are coated by any of the following methods: (i) coating the inner surface of the suppository mold with the coating material before molding of the suppository, (ii) dipping the suppository in the liquid coating material, (iii) molding or casting the coating material around the suppository, or (iv) coating by some other suitable method. The coating can comprise, but is not limited to, fatty or oleaginous bases such as cocoa butter, hard fat and hydrogenated vegetable oil, waxes, water-soluble or water-miscible bases such as polyethylene glycols, glycol-surfactant combinations and polyoxyethylene sorbitan fatty acid esters, and combinations thereof. Furthermore, coated suppositories can be in the form of rectal capsules where a free fatty acid containing core is covered by water-soluble compound such as gelatin.

In some embodiments, the composition may further comprise cocoa butter, coconut oil, glycerinated gelatin, hydrogenated oil, polyethylene glycol (PEG) or a fatty acid ester of PEG.

In some embodiments, commercially available materials suitable for coating according to the present invention include Suppocire™ (Gattefosse) lipophilic bases, a semi-synthetic vegetable based oil base available in several grades including but not limited to Suppocire™ AS, AS2X, NA; Novata™ (Henkel Int.) including Novata A, Novata B, and Novata BC; Witepsol™ (Dynamit Nobel Ab) such as Witepsol™ H5, H12, H15, H32, H35, W25, W31, W32, W32, W35, and W45; Massa Estarinum™ (SASOL), incl. Massa Estarinum™ preferably of the grades B, BC,E and 299; Japocire™ and Ovucire™. The coatings of the present invention may suitably comprise any of the above-mentioned materials as base. Hydrophilic waxes can also be used in the invention, such as the polyethylene glycols (e.g. PEG 1500, PEG 3000, PEG 4000 and mixtures thereof). Suppocire AP, is an amphiphilic base comprising saturated polyglycolyzed glycerides. Mixtures of the above are as well useful in the invention. In some embodiments, hard fat (e.g. suitable Suppocire grade material) constitutes a dominant component of the coating material. By varying the relative amounts of components in the coat, desired characteristics can be adjusted, such as the softening time and disintegration time. A higher amount of hydrophobic waxes such as beeswax generally will increase softening times as well as disintegration times.

The coating material preferably also includes a lubricant such as but not limited to glyceryl dibhenate. In some embodiments, one or more pharmaceutically active ingredients may be added to the coating, such as but not limited to a locally acting anesthetic and/or analgesic such as but not limited to lidocaine, prilocaine, benzocaine, chloroprocaine, bupivacaine, levobupivacaine or other local anesthetic of the amoniester or aminoamide type, or any combination thereof.

Other pharmaceutical compounds may be included in the coating, especially when rapid local delivery is desired, such as but not limited to hydrocortisone. Smaller suppositories for pediatric use are also within the scope of the invention. Depending on the desired dose, total size of the suppository, or rate of release of the therapeutic composition, coating materials may be modified.

A common size of molded or kneaded suppositories for adult use may be in the range of about 2-3 mL, such as about 2.0 mL, about 2.2 mL or about 2.5 mL. Depending on the excipient composition, this would generally correspond to a weight of the uncoated core in the range of about 1.5 to about 3 g, accordingly, the suppositories according to the invention are suitably in said weight range, such as having a core of about 1.8 g, about 2.0 g, about 2.2 g or about 2.5 g. A suitable size for pediatric suppositories would generally be about half the above size, such as in the range of 0.5-1.5 mL, e.g. about 0.5 mL, about 0.8 mL, about 1.0 mL, about 1.2 or about 1.5 mL. Common thickness of the coating layer covering the soft suppository core is about 0.1 to 3 mm.

Coating materials may comprise one or more fatty acid with a chain length ranging from four to 36 carbon atoms, such as a chain length in the range of 4 to 24, or a chain length in the range of 8 to 24 carbons. It is preferable that one or more fatty acids comprise the mixture of fatty acids, which can be derived from suitable natural lipid material such as oils of animal or vegetative origin, fractions thereof, or any combination thereof.

Coating materials may comprise unsaturated fatty acids such as palmitoleic acid (16:1 n-7), cis-vaccenic acid (18:1 n-7), oleic acid (18:1 n-9), elaidic acid (18:1), linoleic acid (18:2 n-6), alpha-linolenic acid (18:3 n-3), gamma-linolenic acid (18:3 n-6), moroctique acid (18:4 n-3), arachidonic acid (20:4 n-6), gadoleic acid (20:1 n-11), gondoic acid (20:1 n-9), cis-11 eicosenoic acid (20:1 n-7), eicosapentaenoic acid (20:5 n-3; EPA), erucic acid (22:1 n-9), cetoleic acid (22:1 n-11), clupanodonic acid (22:5 n-3) and docosahexaenoic acid (22:6 n-3; DHA). Vegetable oils used as raw materials for the fatty acids of the invention may include safflower oil, corn oil, almond oil, sesame oil, soybean oil, linseed oil, rapeseed oil, grape seed oil, sunflower oil, wheat germ oil, hemp oil, or any combination thereof. In some embodiments, the fatty acids are derived from oil material which is pharmaceutically acceptable and defined according to Pharmacopoeia standards (pharmaceutical grade oils). Such oils may include marine omega oils such as Omega-3 Fish Oil (Lysi, Iceland).

In some embodiments, the suppository dosage form of the invention may include an excipient oil component such as a triacylglyceride oil, to reduce discomfort during therapeutic administration as related to bodily functions such as bowel movements. The excipient oil may be composed to ensure the desired melting point of the overall composition of the dosage form.

Although the coating may allow preservative-free suppositories, the coating may comprise anti-oxidants, such as but not limited to butylated hydroxyanisole (BHA), butylated hydroxytoluene (BHT), ascorbic acid or a salt thereof, a sulfatide salt, citric acid, propyl gallate, alpha-tocopherol, ascorbyl palmitate, or any combination thereof. In some embodiments, the coating may include preservatives such as any of those of the group consisting of benzoic acid or derivatives thereof, including of Ci 6-alkyl-p-hydroxy-benzoic acids, such as methyl-p-hydroxy-benzoic acid, ethyl-p-hydroxy-benzoic acid, propyl-p-hydroxy-benzoic acid, butyl-p-hydroxy-benzoic acid, or any combination thereof.

In some embodiments, the coating may comprise further preservative agents, such as any of those of the group consisting of benzoic acid or derivatives thereof, including of Ci_6-alkyl-p-hydroxy-benzoic acids, such as methyl-p-hydroxy-benzoic acid, ethyl-p-hydroxy-benzoic acid, propyl-p-hydroxy-benzoic acid, butyl-p-hydroxy-benzoic acid, or any combination thereof.

In certain embodiments, the preservative is a mixture of methyl-p-hydroxy-benzoic acid and propyl-p-hydroxy-benzoic acid. The preservatives may be present in the coating formulation in a concentration of about 0.05-0.2% by weight, or other concentrations that do not impair the activity of the lipids.

The exosome formulations described here are useful in treating inflammatory and autoimmune disorders in an individual. In certain embodiments, the inflammatory or autoimmune disorder comprises an inflammatory or autoimmune disorder of the gastrointestinal tract. In certain embodiments, inflammatory or autoimmune disorder of the gastrointestinal tract is inflammatory Bowel disease. In certain embodiments, inflammatory or autoimmune disorder of the gastrointestinal tract is inflammatory Chron's disease. In certain embodiments, inflammatory or autoimmune disorder of the gastrointestinal tract is colitis. In certain embodiments. In certain embodiments, the individual is human individual. In certain embodiments, the individual is cow, horse, sheep, goat, pig, cat or dog.

The exosome formulations described here are useful for reducing inflammation in an individual. In certain embodiments, the inflammation is of the gastrointestinal tract. The exosome formulations described here are useful for reducing neutrophil accumulation or activation in the gastrointestinal tract in an individual. In certain embodiments, the individual is human individual. In certain embodiments, the individual is cow, horse, sheep, goat, pig, cat or dog.

In certain embodiments, a therapeutic dose of the exosome compositions comprise about 1 billion exosome particles to about 2,000 billion exosome particles. In certain embodiments, a therapeutic dose of the exosome compositions comprise about 100 billion exosome particles to about 250 billion exosome particles, about 100 billion exosome particles to about 500 billion exosome particles, about 100 billion exosome particles to about 750 billion exosome particles, about 100 billion exosome particles to about 1 billion exosome particles, about 100 billion exosome particles to about 1,250 billion exosome particles, about 100 billion exosome particles to about 1,500 billion exosome particles, about 100 billion exosome particles to about 2,000 billion exosome particles, about 250 billion exosome particles to about 500 billion exosome particles, about 250 billion exosome particles to about 750 billion exosome particles, about 250 billion exosome particles to about 1 billion exosome particles, about 250 billion exosome particles to about 1,250 billion exosome particles, about 250 billion exosome particles to about 1,500 billion exosome particles, about 250 billion exosome particles to about 2,000 billion exosome particles, about 500 billion exosome particles to about 750 billion exosome particles, about 500 billion exosome particles to about 1 billion exosome particles, about 500 billion exosome particles to about 1,250 billion exosome particles, about 500 billion exosome particles to about 1,500 billion exosome particles, about 500 billion exosome particles to about 2,000 billion exosome particles, about 750 billion exosome particles to about 1 billion exosome particle, about 750 billion exosome particles to about 1,250 billion exosome particles, about 750 billion exosome particles to about 1,500 billion exosome particles, about 750 billion exosome particles to about 2,000 billion exosome particles, about 1 billion exosome particles to about 1,250 billion exosome particles, about 1 billion exosome particles to about 1,500 billion exosome particles, about 1 billion exosome particles to about 2,000 billion exosome particles, about 1,250 billion exosome particles to about 1,500 billion exosome particles, about 1,250 billion exosome particles to about 2,000 billion exosome particles, or about 1,500 billion exosome particles to about 2,000 billion exosome particles. In certain embodiments, a therapeutic dose of the exosome compositions comprise about 100 billion exosome particles, about 250 billion exosome particles, about 500 billion exosome particles, about 750 billion exosome particles, about 1 billion exosome particle, about 1,250 billion exosome particles, about 1,500 billion exosome particles, or about 2,000 billion exosome particles. In certain embodiments, a therapeutic dose of the exosome compositions comprise at least about 100 billion exosome particles, about 250 billion exosome particles, about 500 billion exosome particles, about 750 billion exosome particles, about 1 billion exosome particle, about 1,250 billion exosome particles, or about 1,500 billion exosome particles. In certain embodiments, a therapeutic dose of the exosome compositions comprise at most about 250 billion exosome particles, about 500 billion exosome particles, about 750 billion exosome particles, about 1 billion exosome particle, about 1,250 billion exosome particles, about 1,500 billion exosome particles, or about 2,000 billion exosome particles. The doses can be formulated for oral administration, rectal administration, or vaginal administration.

### Methods of Making Rectal or Vaginal Delivery Systems

In certain embodiments, also described herein are methods of making oral delivery systems comprising admixing a live cell population with a matrix described herein and contacting the matrix:live cell population composition with an enteric coating. In certain embodiments, the matrix:live cell composition is added to a capsule before an enteric coating. In certain embodiments, also described herein are methods of making oral delivery systems comprising admixing a live cell population with a matrix comprising agar described herein and contacting the matrix:live cell population composition with an enteric coating comprising a copolymer of methacrylic acid.

In another aspect a method of making a composition of mammalian exosomes formulated for oral delivery comprises admixing a plurality of mammalian exosomes with a matrix comprising from about 1.0% to about 2.0% methyl cellulose and about 1.0% to about 2.0% alginate gel to provide a cell-matrix, and applying an enteric coating to cell-matrix. In certain embodiments, the matrix comprises from about 1.3% to about 1.8% methylcellulose and from about 1.3% to about 1.8% alginate gel. In certain embodiments, the matrix comprises about 1.5% methylcellulose and about 1.5% alginate gel. In certain embodiments, the methyl cellulose and alginate gel are present at a ratio of about 1:1. In certain embodiments, the enteric coating comprises methyl methacrylate-methacrylic acid copolymer (1:1). In certain embodiments, the plurality of mammalian exosomes comprises human exosomes. In certain embodiments, the plurality of mammalian exosomes comprises non-human exosomes.

In certain embodiments, also described herein are methods of making rectal or vaginal delivery systems comprising admixing with a matrix described herein and contacting the matrix:exosome preparation with a lipophilic or hydrophilic coating. In certain embodiments, the matrix:exosome preparation is added to a capsule before a lipophilic or hydrophilic coating. In certain embodiments, also described herein are methods of making rectal or vaginal delivery systems comprising admixing an exosome preparation with a matrix comprising agar described herein and contacting the matrix:exosome preparation with a lipophilic or hydrophilic coating comprising an excipient or copolymer of methacrylic acid.

In certain embodiments, also described herein are methods of making rectal or vaginal delivery systems comprising admixing an exosome preparation with a matrix comprising agar described herein and contacting the matrix:exosome preparation with a lipophilic or hydrophilic coating comprising an excipient or cocoa butter, coconut oil, glycerinated gelatin, hydrogenated oil, polyethylene glycol (PEG), or a fatty acid ester of PEG.

In certain embodiments, also described herein are methods of making an oral delivery systems comprising admixing an exosome preparation with a matrix described herein and contacting the matrix:exosome preparation with an enteric coating. In certain embodiments, the matrix:exosome preparation is added to a capsule before an enteric coating. In certain embodiments, also described herein are methods of making rectal delivery systems comprising admixing an exosome preparation with a matrix comprising agar described herein and contacting the matrix:exosome preparation with an enteric coating comprising an excipient or copolymer of methacrylic acid.

In another aspect a method of making a preparation of exosomes formulated for rectal or vaginal delivery comprises admixing the preparation of exosomes with a matrix comprising from about 0.5% to about 3.0% methyl cellulose and about 0.5% to about 3.0% alginate gel to provide a cell-matrix, and applying a lipophilic or hydrophilic coating to cell-matrix. In certain embodiments, the matrix comprises from about 1.0% to about 2.0% methylcellulose and from about 1.0% to about 2.0% alginate gel. In certain embodiments, the matrix comprises from about 1.3% to about 1.8% methylcellulose and from about 1.3% to about 1.8% alginate gel. In certain embodiments, the matrix comprises about 1.5% methylcellulose and about 1.5% alginate gel. In certain embodiments, the methyl cellulose and alginate gel are present at a ratio of about 1:1. In certain embodiments, the lipophilic or hydrophilic coating comprises methyl methacrylate-methacrylic acid copolymer (1:1).

### Certain Embodiments

1. A composition for non-parenteral and/or rectal or vaginal delivery of mammalian exosomes, the composition comprising a lipophilic or hydrophilic coating surrounding a plurality of mammalian exosomes admixed with a matrix in solid form, the matrix comprising a matrix comprising about 0.5% to about 3.0% methyl cellulose and about 0.5% to about 3.0% alginate gel.
2. The composition of embodiment 1, wherein the matrix comprises about a matrix comprises about 1.0% to about 2.0% methyl cellulose and about 1.0% to about 2.0% alginate gel.
3. The composition of embodiment 1, wherein the matrix comprises about 1.3% to about 1.8% methylcellulose and about 1.3% to about 1.8% alginate gel.
4. The composition of embodiment 1, wherein the matrix comprises about 1.5% methylcellulose and about 1.5% alginate gel.
5. The composition of any one of embodiments 1 to 4, wherein the matrix comprises a ratio of methyl cellulose to alginate gel of about 1:1.
6. The composition of any one of embodiments 1 to 5, wherein the exosomes are mesenchymal stem cell exosomes or hematopoietic stem cell exosomes.
7. The composition of embodiment 6, wherein the exosomes are hematopoietic stem cell exosomes.
8. The composition of embodiment 6, wherein the exosomes are mesenchymal stem cell exosomes.
9. The composition of embodiment 8, wherein the mesenchymal stem cells are type I mesenchymal stem cells.
10. The composition of embodiment 8, wherein the mesenchymal stem cells are type II mesenchymal stem cells.
11. The composition of any one of embodiments 8 to 10, wherein the exosomes comprise a protein or nucleic acid selected from the list consisting of CD81, CD63, CD9, HSP60,HSP70, HSP90, VEGF, PGE2, and combinations thereof.
12. The composition of any one of embodiments 8 to 10, wherein the exosomes comprise a nucleic acid selected from the list consisting of miRNA Let7, 155,146, 122, and combinations thereof.
13. The composition of any one of embodiments 1 to 12, wherein the mammalian exosomes possess a diameter from about 30 nanometers to about 150 nanometers.
14. The composition of any one of embodiments 1 to 13, wherein the mammalian exosomes are human exosomes.
15. The composition of any one of embodiments 1 to 13, wherein the mammalian exosomes are canine, feline, bovine, equine, ovine, or porcine exosomes.
16. The composition of any one of embodiments 1 to 15, wherein the composition is formulated for rectal delivery.
17. The composition of any one of embodiments 1 to 15, wherein the composition is formulated for vaginal delivery.
18. The composition of any one of embodiments 1 to 16, wherein the formulation does not swell upon contact with an aqueous medium.
19. The composition of any one of embodiments 1 to 18, wherein the lipophilic or hydrophilic coating dissolves at a normal body temperature, or at 36° C. and above.
20. The composition of any one of embodiments 1 to 19, wherein the lipophilic or hydrophilic coating consists essentially of cocoa butter, coconut oil, glycerated gelatin, hydrogenated oil, polyethylene glycol (PEG), a fatty acid ester of PEG, or Gattefosse lipophilic bases.
21. The composition of any one of embodiments 1 to 19, wherein the lipophilic or hydrophilic coating comprises cocoa butter, coconut oil, glycerated gelatin, hydrogenated oil, polyethylene glycol (PEG), a fatty acid ester of PEG, or Gattefosse lipophilic bases.

### EXAMPLES

The following illustrative examples are representative of embodiments of compositions and methods described herein and are not meant to be limiting in any way.

### Example 1—Collection of Mammalian Exosomes from Mesenchymal Stem Cells

### MSC Exosome Collection

MSCs are incubated at 37° C. in a humidified atmosphere with 5% CO₂. Adherent cells are allowed to expand until they reached about 80% confluence. Morphology of isolated and expanded MSC cells is assessed during the cell culture period using light microscopy and FACS analysis.

For exosome production and harvest, cells are cultivated in optimal growth medium up to 72 h (<2% exosome-free). After the cultivation and subsequent priming period cell supernatant is collected and exosomes are isolated using a Fab-TACS® Exosome Isolation Kit, (IBA Lifesciences, ThermoFisher Scientific, CA).

Enrichment of CD9 and CD81 positive exosomes from MSC cell culture supernatant is performed using the respective Fab-TACS® Exosome Isolation Kits according to the included manuals. Briefly, the MSC cell culture supernatant is centrifuged at 3000×g for 10 min. The supernatant is filtrated through a 0.2 μm polyether-sulfone filter. Elution Buffer and PBS are filtered through a cellulose-acetate filter (0.2 μm). After draining the storage buffer, columns are loaded with Fab-Streps. Filtrated culture media are transferred to the column in 1 ml steps. After washing, filtrated Biotin Elution Buffer is applied to the matrix to collect exosomes. Exosome size is analyzed with the NanoSight LM10 instrument (Malvern Instruments) and data are processed using Nanoparticle Tracking Analysis [NTA] software 2.3. Protein content is determined by standard protein assays or by Western Blot using monoclonal detection antibodies for CD9, Alix, and CD63.

### Example 2—Stability of Exosomes at Different pH and Under Different Gastrointestinal Conditions

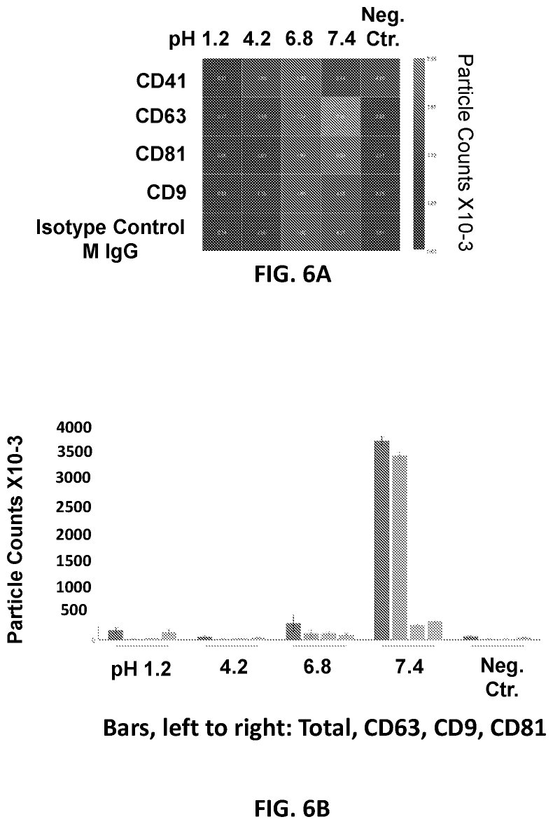

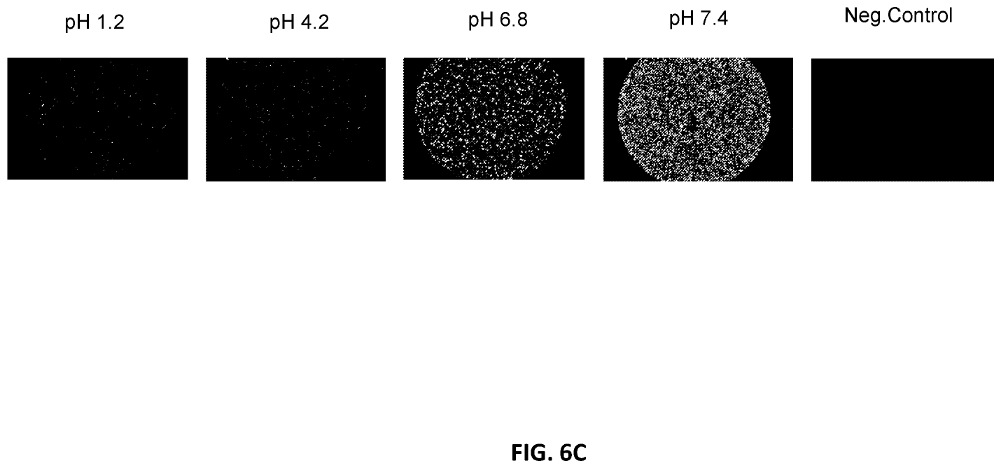

Exosomes from human adult tissue derived mesenchymal stem cells [5 billion particles] were loaded into the oral formulation (0.67% w/v alginate and 0.67% w/v methylcellulose and coated with a methyl methacrylate-methacrylic acid copolymer (1:1) enteric coating). The enteric capsules were then treated similarly to expected pH changes of the capsules traveling down the oral cavity and into the ileum and colon. Thus, a 2 hr pH=1.2 incubation was followed by 30 min at pH 4.2, 30 min at pH 6.8 and lastly >2 hr pH=7.4 wherein the exosomes were recovered, concentrated 10× and measured. FIGS. 6A and 6B show, that the enteric capsules did not release their payloads until after reaching the neutralized >6.8 pH. FIG. 6B shows the number of particles recovered and stained for exosome specific markers CD63, CD81, and CD9 from each sample measured with a NanoSight NS300 and analyzed with the ExoViewAnalyzer 3.0 [R100, NanoView Biosciences, MA]. FIG. 6C shows that the recovered exosomes were intact by immunofluorescence microscopy.

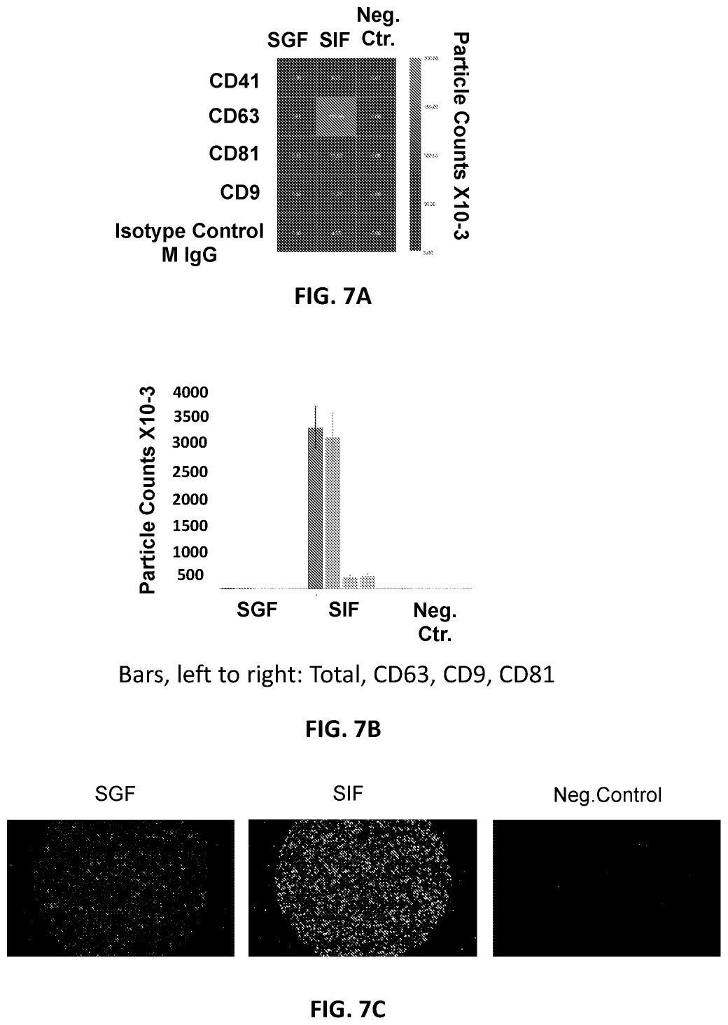

Exosomes [5 billion particles] containing enteric coated capsules were treated similarly to expected pH changes of the capsules traveling down the oral cavity and into the ileum and colon. Thus, a 2 hr pH=1 (USP [US Pharmacopeia] standard, Simulated gastric fluid, SIG) incubation was followed by a 2 hr pH=7.5 neutralization in Simulated Intestinal Fluid (USP [US Pharmacopeia], SIF) wherein the cells were recovered and assayed as described above. FIGS. 7A and 7B show that, the enteric capsules did not release their payloads until after reaching the neutralized SIF. The figure shows the number of particles recovered and stained for exosome specific markers CD63 (red), CD81 (blue), and CD9 (green) from each sample (IgG isotype negative control, gray) measured with a NanoSight NS300 and analyzed with the ExoViewAnalyzer 3.0 [R100, NanoView Biosciences, MA]. FIG. 7C shows that the recovered exosomes were intact by immunofluorescence microscopy.

The recovery of exosomes ad pHs replicating the lower GI tract after incubation at high pH (mimicking the stomach and upper GI), as well as incubation in simulated gastric fluid is surprising as the described exosome compositions with an alginate and methylcellulose matrix possess alginate gel pore sizes ranging from 5-150 nm [25-200 kDa], while the exosomes are about 70-150 nM. Nevertheless, the matrix appears to hold the exosomes in a pH dependent manner allowing them to pass intact through the low pH/high-proteolytic environment of the upper GI and deliver them to the colon.

### Example 3—Oral Exosome Formulations Treat a Mouse Model of IBD

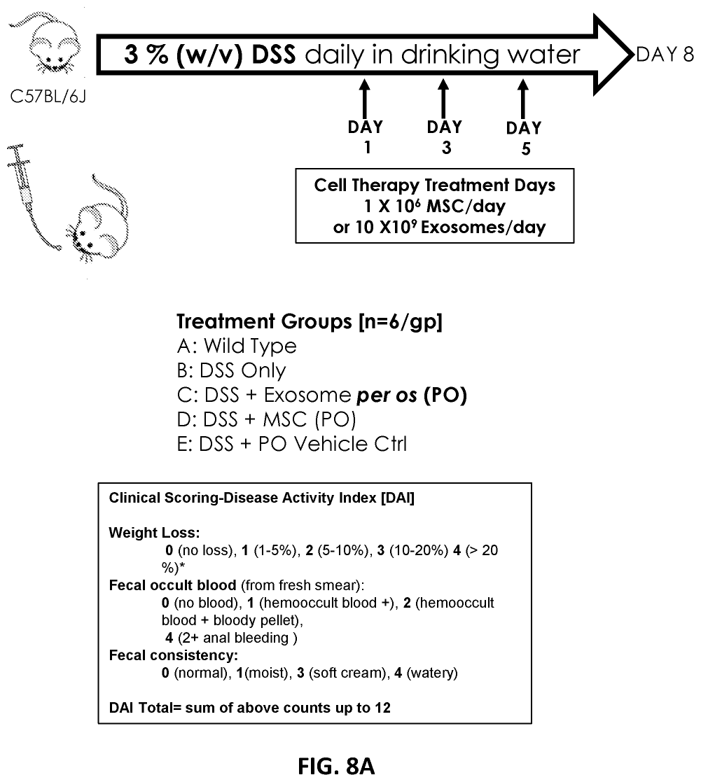

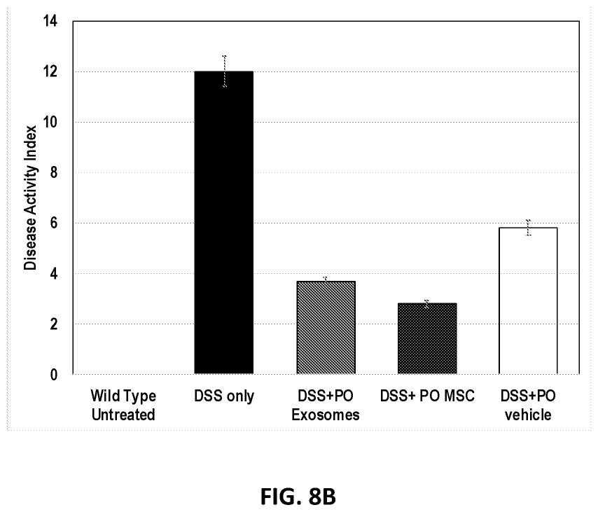

The efficacy of the oral exosome formulations described was tested in the dextran sulfate sodium (DSS) induced colitis model. This model is an established and validated model of inflammatory bowel disease (IBD) and mirrors human IBD. See Li et al. Mesenchymal stem cells and acellular products attenuate murine induced colitis.” Stem Cell Research and Therapy. (2020) 11:515. In these experiments, colitis is induced in mice using DSS administered at a concentration of 3% wt/vol in drinking water. Briefly, DSS (MW 40,000, Sigma-Aldrich, St. Louis, MO) is administered in drinking water daily throughout the study period. As shown in FIG. 8A, for each study, mice (n=6 per group) were randomly assigned to the following groups: (a) untreated wild type control group; (b) DSS treatment only; (c) DSS per os (PO) exosomes [10 billion particles] administration; (d) DSS PO MSC [1M cells] administration; and (e) DSS and oral formula vehicle control. Mice were monitored daily for body weight, clinical signs, stool consistency and color daily. On days 1, 3, and 5 of the study (with DSS administration initiated on day 0), vehicle, exosomes and MSC were administered orally as indicated. Clinical scoring was done using a modified scoring matrix described in FIG. 8A. Fecal occult blood was measured using a test kit (Fisher Healthcare, Houston, TX). As shown in FIG. 8B, this treatment with orally formulated exosomes led to a significant reduction in symptoms associated with DSS induced colitis in mice.

### Example 4—Oral Exosome Formulations Retain Broadly Anti-Inflammatory Properties

Preclinical diseased animal studies demonstrated that the primary therapeutic benefit of the exosome therapies is through attenuation of neutrophil infiltrates and neutrophil-released inflammatory factors. A potency assay to measure specific inflammatory factors released by activated neutrophils was designed. Neutrophils were sourced from an established ATCC human neutrophil-like cell line [HL60] that can be banked and standardized. These cells were co-cultured alone (negative control), with anti-inflammatory MSCs or exosomes (positive control cells, starting exosome product), the recovered and 10× concentrated oral exosome products from the pH studies, and the effect on specific inflammatory factors expression was assessed by RT-qPCR assay.

Briefly, HL60 cells were cultured in RPMI, followed by neutrophil-like induction of the cells accomplished by addition of 1.3% DMSO for three days. Neutrophil-like HL60 cells @2.5 million cells/mL were then transferred to 24 well plates with adherent human BM MSC [250,000 cells-0.1× as positive control] or 5 billion EVs and these were activated or not [negative control] with 10 ug/mL LPS overnight. HL60 cells were subsequently collected and RNA was extracted with miRNAeasy kit [Qiagen, MD] prior to real time qPCR with human gapdh as the normalizing housekeeping gene on a BioRad CFX96 [BioRad, CA].

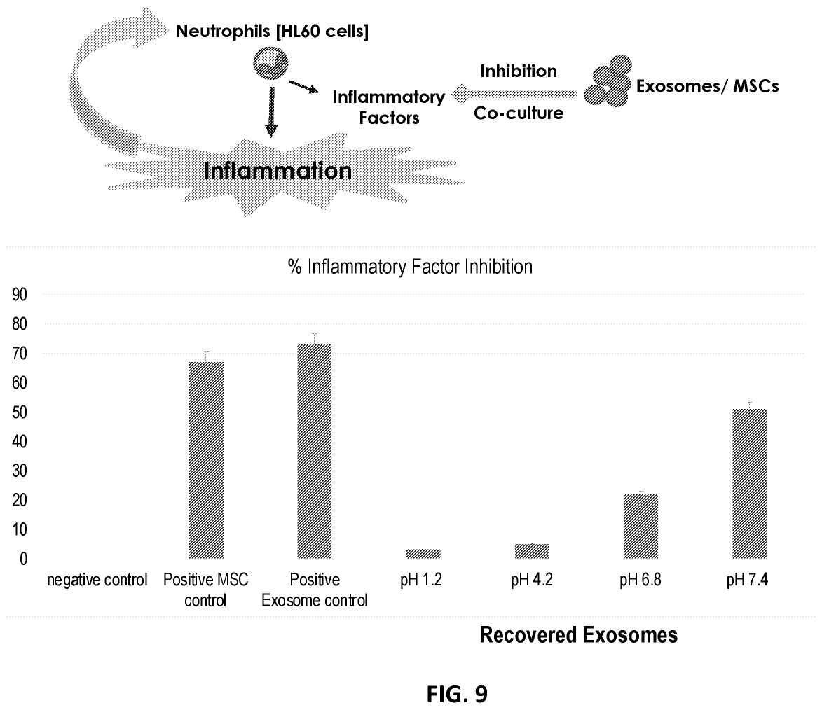

Exemplary data from such an assay is shown in FIG. 9 and indicates that a key mechanism of action of the described exosome is the inhibition of neutrophil activity and thus inflammation generally.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

What is claimed is:

1. A composition for oral delivery of mammalian exosomes, the composition comprising a plurality of mammalian exosomes admixed with a matrix and surrounded by an enteric coating, the matrix comprising about 0.1% to about 5.0% methyl cellulose and about 0.1% to about 5.0% alginate.

2. The composition of claim 1, wherein the matrix comprises about 0.5% to about 1.0% methyl cellulose and about 0.5% to about 1.0% alginate.

3. The composition of claim 1, wherein the matrix comprises about 0.25% to about 0.75% methyl cellulose and about 0.25% to about 0.75% alginate.

4. The composition of claim 1, wherein the matrix comprises about 0.5% to about 3.0% methyl cellulose and about 0.5% to about 3.0% alginate.

5. The composition of claim 1, wherein the matrix comprises about 1.0% to about 2.0% methyl cellulose and about 1.0% to about 2.0% alginate.

6. The composition of claim 1, wherein the matrix comprises about 1.3% to about 1.8% methylcellulose and about 1.3% to about 1.8% alginate.

7. The composition of claim 1, wherein the matrix comprises about 1.5% methylcellulose and about 1.5% alginate.

8. The composition of any one of claims 1 to 7, wherein the matrix comprises a ratio of methyl cellulose to alginate of about 1:1.

9. The composition of any one of claims 1 to 8, wherein the exosomes are mesenchymal stem cell exosomes or hematopoietic stem cell exosomes.

10. The composition of claim 9, wherein the exosomes are hematopoietic stem cell exosomes.

11. The composition of claim 9, wherein the exosomes are mesenchymal stem cell exosomes.

12. The composition of claim 11, wherein the mesenchymal stem cells are type I mesenchymal stem cells.

13. The composition of claim 11, wherein the mesenchymal stem cells are type II mesenchymal stem cells.

14. The composition of any one of claims 11 to 13, wherein the exosomes comprise a protein or nucleic acid selected from the list consisting of CD81, CD63, CD9, HSP60, HSP70, HSP90, VEGF, PGE2, and combinations thereof.

15. The composition of any one of claims 1 to 13, wherein the exosomes comprise a nucleic acid selected from the list consisting of miRNA Let7, 155,146, 122, and combinations thereof.

16. The composition of any one of claims 1 to 15, wherein the mammalian exosomes possess a diameter from about 30 nanometers to about 150 nanometers.

17. The composition of any one of claims 1 to 16, wherein the mammalian exosomes are human exosomes.

18. The composition of any one of claims 1 to 16, wherein the mammalian exosomes are canine, feline, bovine, equine, ovine, or porcine exosomes.

19. The composition of any one of claims 1 to 18, wherein the enteric coating comprises methyl methacrylate-methacrylic acid copolymer (1:1).

20. The composition of any one of claims 1 to 18, wherein the enteric coating consists essentially of methyl methacrylate-methacrylic acid copolymer (1:1).

21. The composition of any one of claims 1 to 20, wherein the composition comprises at least 100 billion exosomes.

22. The composition of any one of claims 1 to 20, wherein the composition comprises from at least 100 billion exosomes to no more than 2,000 billion exosomes.

23. The composition of any one of claims 1 to 22, wherein greater than 40% or more intact exosomes compared to the starting amount are recovered from the composition after a 2 hour incubation at pH 1.2.

24. The composition of any one of claims 1 to 22, wherein greater than 50% or more intact exosomes compared to the starting amount are recovered from the composition after a 2 hour incubation at pH 1.2.

25. The composition of any one of claims 1 to 22, wherein greater than 60% or more intact exosomes compared to the starting amount are recovered from the composition after a 2 hour incubation at pH 1.2.

26. A method of reducing neutrophil accumulation or activation of neutrophils in the gastrointestinal tract of an individual comprising administering the composition of any one of claims 1 to 25, orally to the individual.

27. A method of treating an inflammatory or autoimmune disorder in an individual comprising administering the composition of any one of claims 1 to 25, orally to the individual.

28. The method of claim 26 or 27, wherein the individual is a human individual.

29. The method of claim 26 or 27, wherein the individual is a dog, a cow, a cat, a pig, a horse, or a sheep.

30. The method of claim 27, wherein the inflammatory or autoimmune disorder is a gastrointestinal inflammatory or autoimmune disorder.

31. The method of claim 27, wherein the gastrointestinal inflammatory or autoimmune disorder comprises inflammatory bowel disease or Crohn's disease.

32. The method of claim 27, wherein the gastrointestinal inflammatory or autoimmune disorder comprises inflammatory bowel disease (IBD).
